(19) United States
(12) Patent Application Publication
(10) Pub. No.: US 2026/0338666 A1
(43) Pub. Date: Oct. 29, 2026

(54) SYSTEM AND METHOD FOR DETERMINING PERFUSED TISSUE VIABILITY

(71) Applicant: The General Hospital Corporation, Boston, MA (US)

(72) Inventors: Conor L. Evans, Boston, MA (US); Yanis Berkane, Boston, MA (US); Mustafa Korkut Uygun, Boston, MA (US); Juan Pedro Cascales Sandoval, Boston, MA (US); Alexandre G. Lellouch, Boston, MA (US); Emmanouil Rousakis, Boston, MA (US)

(21) Appl. No.: 19/116,076

(22) PCT Filed: Sep. 28, 2023
(86) PCT No.: PCT/US2023/075328

§ 371 Date: Mar. 27, 2025

Related U.S. Application Data
(60) Provisional application No. 63/377,519, filed on Sep. 28, 2022.

(51) Int. Cl. G01N 15/1434; C12M 1/00; C12M 1/34; C12M 3/00; G01N 15/01; G01N 15/10; G01N 33/00

(52) U.S. Cl. CPC G01N 15/1436; C12M 21/08; C12M 23/22; C12M 29/10; C12M 41/34; G01N 15/01; G01N 33/0031; G01N 2015/1006

(57) ABSTRACT

The disclosure provides perfusion systems and methods for continuous or intermittent perfusion to monitor and extend the viability of tissue for transplants. Intermittent perfusion involves generating perfusion cycles which alternate between baseline and high oxygen gas partial pressure in the circulating perfusate. The system comprises a perfusion fluid source and an inflow conduit in fluid communication with the perfusion fluid source and a tissue sample, wherein the inflow conduit is configured to deliver perfusion fluid to the tissue sample. The system further comprises an outflow conduit in fluid communication with the tissue sample, wherein the outflow conduit is configured to carry perfusion fluid away from the tissue sample. A first gas sensor is in fluid communication with the inflow-conduit and measures a. concentration of a gas in perfusion fluid, in the inflow conduit, and a second gas sensor is in contact with the tissue sample and measures a concentration of a gas in the sample.

### CROSS REFERENCE TO RELATED APPLICATIONS

The present application is based on, claims priority to, and incorporates herein by reference in its entirety for all purposes, U.S. Patent Application No. 63/377,519, filed Sep. 28, 2022.

### STATEMENT REGARDING FEDERALLY SPONSORED RESEARCH

This invention was made with government support under award numbers FA9550-20-1-0063 from the Air Force Office of Scientific Research, and NSF1941543 from the National Science Foundation, and W81XWH1910440 from the U.S. Department of Defense. The government has certain rights in the invention.

### BACKGROUND

Perfusion machine techniques have undergone a remarkable revival in recent years, and ex-vivo perfusion is beginning to routinely supplant static preservation for solid organs such as the kidney, liver, or heart [Ref. 1-4]. Normothermic Machine Perfusion is becoming a standard in the United States for extended criteria donor livers, allowing assessment of the organ's quality before transplantation [Ref. 5]. Similarly, hypothermic machine perfusion has progressively supplanted static cold storage for kidney preservation before transplantation [Ref. 6]. On the other hand, complex reconstructive surgeries such as Vascularized Composite Allotransplantations (VCAs) are becoming of interest to the transplant community [Ref. 7-9]. Recent results of VCA are awe-inspiring from a functional, aesthetic, and social point of view, but long-term outcomes are still unsatisfactory, mainly due to chronic rejection [Ref. 10, 11]. The muscle component of these allografts is susceptible to ischemia, while the skin and bone marrow components are an immunogenic challenge. One of the key strategies to mitigate VCA immune rejection is to reduce the occurrence of ischemia-reperfusion injuries [Ref. 12, 13]. The use of machine perfusion is particularly valuable for preserving delicate allografts [Ref. 14], as demonstrated in solid organs.

The monitoring of perfused organs generally involves biochemical measurements, vascular pressures, and weight, which reflects edema [Ref. 15, 16]. However, this monitoring is not continuous, and a deterioration of biochemical results (such as an increase in lactate and kaliemia, or a decrease in partial pressure of oxygen (PO₂)) may indicate cellular suffering and suboptimal preservation [Ref. 14]. Tissue oxygenation is a crucial parameter for monitoring perfused organs ex-vivo. Under-oxygenation can be considered a partial failure of the perfusion, whereas uncontrolled overexposure to oxygen can lead to damage caused by free reactive oxygen species [Ref. 17].

Therefore, there is a need for improved systems and methods for determining viability and quality of a perfused tissue sample.

### SUMMARY

The present disclosure provides systems and methods that overcome the aforementioned drawbacks by providing a system and method for tissue sample perfusion including gas inflow and sample sensors to continuously monitor perfusion.

In one aspect, the present disclosure provides a perfusion system for a tissue sample, wherein the system comprises: a perfusion fluid source; an inflow conduit in fluid communication with the perfusion fluid source and the sample, the inflow conduit being configured to deliver perfusion fluid to the sample; an outflow conduit in fluid communication with the sample, the outflow conduit being configured to carry perfusion fluid away from the sample; a first gas sensor in fluid communication with the inflow conduit, the first gas sensor measuring a concentration of a gas in the perfusion fluid in the inflow conduit; and a second gas sensor contacting the sample, the second gas sensor measuring a concentration of a gas in the sample.

In one embodiment of the perfusion system, the second gas sensor comprises: a photoluminescent oxygen-sensitive probe in contact with the sample; a photon source configured to direct photons at the photoluminescent oxygen-sensitive probe; a photodetector configured to detect light emitted from the photoluminescent oxygen-sensitive probe when the photon source directs photons at the photoluminescent oxygen-sensitive probe; and a controller in electrical communication with the photon source and the photodetector, the controller being configured to execute a program stored in the controller to calculate a concentration of oxygen adjacent to the photoluminescent oxygen-sensitive probe from an electrical signal received from the photodetector. In one embodiment of the perfusion system, the photoluminescent oxygen-sensitive probe is a formulation having an emission that provides tissue oxygen partial pressure (pO₂). In one embodiment of the perfusion system, the photoluminescent oxygen-sensitive probe comprises a polymeric material impregnated with a porphyrin. In one embodiment of the perfusion system, the photoluminescent oxygen-sensitive probe comprises a polymer impregnated with a phosphorescent meso-unsubstituted porphyrin.

In one embodiment of the perfusion system, the second gas sensor comprises: an oxygen-sensitive probe configured for insertion into the sample; and a controller in electrical communication with the oxygen-sensitive probe, the controller being configured to execute a program stored in the controller to calculate a concentration of oxygen adjacent the oxygen-sensitive probe from an electrical signal received from the oxygen-sensitive probe. In one embodiment of the perfusion system, the second gas sensor provides tissue oxygen partial pressure (pO₂).

In one embodiment, the perfusion system further comprises: a controller in electrical communication with the first gas sensor and the second gas sensor, the controller being configured to execute a program stored in the controller to receive electrical signals from the first gas sensor and the second gas sensor and to calculate a concentration of oxygen adjacent the first gas sensor and/or the second gas sensor. In one embodiment of the perfusion system, the first gas sensor comprises: a material configured to undergo a change in response to oxygen partial pressure (pO₂); a sensor head contacting the material and configured to detect the change of the material; and a controller in electrical communication with the sensor head, the controller being configured to execute a program stored in the controller to calculate the oxygen partial pressure adjacent the material.

In one embodiment, the perfusion system further comprises: a transparent membrane forming an outer layer of the material; a layer of a polymeric material embedded with a metalloporphyrin adjacent to the transparent membrane; and a scattering layer in contact with the layer of the polymeric material and a surface of the sample. In one embodiment of the perfusion system, the first gas sensor comprises a flow cell containing the material. In one embodiment of the perfusion system, the change in the material includes a change in phosphorescence. In one embodiment of the perfusion system, the sensor head includes a plurality of light emitting diodes (LEDs) and a photodiode. In one embodiment of the perfusion system, the sensor head further includes a temperature sensor.

In one embodiment of the perfusion system, the second gas sensor comprises: a material configured to undergo a change in response to oxygen pressure; and a sensor head contacting the material and configured to detect the change of the material; and a controller in electrical communication with the sensor head, the controller being configured to execute a program stored in the controller to calculate the oxygen partial pressure adjacent the material. In one embodiment of the perfusion system, in the second oxygen sensor, the material is placed on an outer surface of the sample. In one embodiment of the perfusion system, the change in the material includes a change in phosphorescence intensity or phosphorescence lifetime. In one embodiment of the perfusion system, the sensor head includes a plurality of light emitting diodes (LEDs) and a photodiode. In one embodiment of the perfusion system, the sensor head further includes a temperature sensor.

In one embodiment, the perfusion system further comprises: a third gas sensor in fluid communication with the outflow conduit, the third gas sensor measuring a concentration of a gas in perfusion fluid in the outflow conduit. In one embodiment of the perfusion system, the third gas sensor comprises: a material configured to undergo a change in response to oxygen partial pressure (pO₂); a sensor head contacting the material and configured to detect the change of the material; and a controller in electrical communication with the sensor head, the controller being configured to execute a program stored in the controller to calculate the oxygen partial pressure adjacent to the material.

In one embodiment, the perfusion system further comprises: a transparent membrane forming an outer layer of the material; a layer of a polymeric material embedded with a metalloporphyrin adjacent to the transparent membrane; and a scattering layer in contact with the layer of the polymeric material and a surface of the sample. In one embodiment of the perfusion system, the third gas sensor includes a flow cell containing the material. In one embodiment of the perfusion system, the change in the material includes a change in phosphorescence intensity or phosphorescence lifetime. In one embodiment of the perfusion system, the sensor head includes a plurality of light emitting diodes (LEDs) and a photodiode. In one embodiment of the perfusion system, the sensor head further includes a temperature sensor.

In one embodiment, the perfusion system further comprises: an oxygenator in fluid communication with the perfusion fluid source; and a pump for circulating the perfusion fluid through the oxygenator, the inflow conduit, and the outflow conduit, wherein the oxygenator controls oxygen level in perfusion fluid flowing towards the sample.

In one embodiment, the perfusion system further comprises: a heat exchanger for adjusting a temperature of the perfusion fluid.

In one embodiment, the perfusion system further comprises: a controller in electrical communication with the oxygenator, the first gas sensor, and the second gas sensor, the controller being configured to execute a program stored in the controller to control oxygen level in perfusion fluid flowing towards the sample based on electrical signals received from the first gas sensor and the second gas sensor.

In one embodiment, the perfusion system further comprises: a controller in electrical communication with the pump, the first gas sensor, and the second gas sensor, the controller being configured to execute a program stored in the controller to control oxygen level in perfusion fluid flowing towards the sample based on electrical signals received from the first gas sensor and the second gas sensor.

In one embodiment, the perfusion system further comprises: a controller in electrical communication with the first gas sensor and the second gas sensor, the controller being configured to execute a program stored in the controller to receive electrical signals from the first gas sensor and the second gas sensor and to activate delivery of the perfusion fluid when a loss of viability is calculated based on the first concentration of the first gas adjacent the first gas sensor and the second concentration of the second gas adjacent the second gas sensor.

In one embodiment, the perfusion system further comprises: a controller in electrical communication with the first gas sensor and the second gas sensor, the controller being configured to execute a program stored in the controller to receive electrical signals from the first gas sensor and the second gas sensor and to deactivate delivery of the perfusion fluid when a calculated viability based on the first concentration of the first gas adjacent the first gas sensor and the second concentration of the second gas adjacent the second gas sensor exceeds a threshold viability value.

In one embodiment, the perfusion system further comprises: a controller in electrical communication with the first gas sensor and the second gas sensor, the controller being configured to execute a program stored in the controller to receive electrical signals from the first gas sensor and the second gas sensor and to intermittently activate or deactivate delivery of the perfusion fluid based on the first concentration of the first gas adjacent the first gas sensor and the second concentration of the second gas adjacent the second gas sensor.

In one embodiment, the perfusion system further comprises: a controller in electrical communication with the first gas sensor and the second gas sensor, the controller being configured to execute a program stored in the controller to activate an alarm when a gas level drops below a defined threshold based on electrical signals received from the first gas sensor and the second gas sensor.

In one embodiment of the perfusion system, the perfusion fluid is an acellular perfusate solution.

In one embodiment of the perfusion system, the first gas sensor provides continuous circulating oxygen values delivered to the perfused sample. In one embodiment of the perfusion system, the second gas sensor provides continuous tissue oxygenation values of the perfused sample.

In another aspect, the present disclosure provides a method for machine perfusion of a tissue sample. The method comprises: (a) providing a tissue sample; (b) delivering a perfusion fluid to the sample via an inflow conduit; (c) measuring a concentration of a gas in the perfusion fluid in the inflow conduit; and (d) measuring a concentration of a gas in the sample. The method can further comprise: (e) measuring a concentration of a gas in the perfusion fluid in an outflow conduit configured to carry perfusion fluid away from the sample.

In one embodiment of the method, step (c) comprises measuring the concentration of the gas in the perfusion fluid in the inflow conduit with a first gas sensor, step (d) comprises measuring the concentration of the gas in the sample with a second gas sensor, and step (e) comprises measuring the concentration of the gas in the perfusion fluid in the outflow conduit with a third gas sensor, wherein at least one of the first gas sensor, the second gas sensor and the third gas sensor comprises an oxygen-sensitive probe.

In one embodiment of the method, step (c) comprises measuring the concentration of the gas in the perfusion fluid in the inflow conduit with a first gas sensor, step (d) comprises measuring the concentration of the gas in the sample with a second gas sensor; and step (e) comprises measuring the concentration of the gas in the perfusion fluid in the outflow conduit with a third gas sensor, wherein at least one of the first gas sensor, the second gas sensor and the third gas sensor comprises a material configured to undergo a change in response to oxygen partial pressure (pO₂).

In one embodiment of the method, step (c) comprises sensing the concentration of the gas in the perfusion fluid in the inflow conduit with the first gas sensor and calculating, in a controller, a concentration of oxygen adjacent to the first gas sensor from an electrical signal transmitted to the controller from the first gas sensor, step (d) comprises measuring the concentration of the gas in the sample with the second gas sensor, and calculating, in the controller, a concentration of oxygen adjacent the second gas sensor from an electrical signal transmitted to the controller from the second gas sensor and, step (e) comprises sensing the concentration of the gas in the perfusion fluid in the outflow conduit with the third gas sensor and calculating, in a controller, a concentration of oxygen adjacent to the third gas sensor from an electrical signal transmitted to the controller from the third gas sensor.

In one embodiment, the method further comprises: monitoring a change in the concentration from the first gas sensor, and at least one of the second gas sensor and third gas sensor; measuring a variation in the oxygen concentration over a period of time; analyzing the variation in the oxygen concentration over the period of time; and estimating a viability of the tissue sample and a perfusion quality.

In one embodiment, the method further comprises: (e) generating a report, using the controller, of oxygen perfusion in the sample. In one embodiment, the method further comprises: (e) controlling an oxygen level in the perfusion fluid flowing towards the sample based on electrical signals received from the first gas sensor and the second gas sensor.

In one embodiment, the method further comprises: (e) activating an alarm when a gas level drops below a defined threshold based on electrical signals received from the first gas sensor and the second gas sensor.

In one embodiment of the method, the tissue sample is a vascular composite allograft. In one embodiment of the method, the vascular composite allograft is at least a portion of a limb, face, larynx, trachea, abdominal wall, genitourinary tissue, uterine tissue, or solid organ, or any combination thereof. In one embodiment of the method, the tissue sample is a donor vascular composite allograft for vascular composite allograft transplantation. In one embodiment of the method, the tissue sample is obtained from a human, a primate, or a pig. In one embodiment of the method, the tissue sample is a fasciocutaneous flap.

In another aspect, the present disclosure provides a perfusion system for a tissue sample, wherein the system comprises: a perfusion fluid source; an inflow conduit in fluid communication with the perfusion fluid source and the sample, the inflow conduit being configured to deliver perfusion fluid to the sample; an outflow conduit in fluid communication with the sample, the outflow conduit being configured to carry perfusion fluid away from the sample; a first gas sensor in fluid communication with the inflow conduit, the first gas sensor measuring a concentration of a first gas in the perfusion fluid in the inflow conduit, a second gas sensor contacting the sample, the second gas sensor measuring a concentration of a second gas in the sample; and a controller in electrical communication with the first gas sensor and the second gas sensor, the controller being configured to execute a program stored in the controller to: (i) receive electrical signals from the first gas sensor and the second gas sensor and to calculate a first concentration of the first gas adjacent the first gas sensor and a second concentration of the second gas adjacent the second gas sensor, and (ii) activate or deactivate delivery of the perfusion fluid flowing towards the sample and/or adapt perfusion parameters based on the first concentration of the first gas adjacent to the first gas sensor and the second concentration of the second gas adjacent to the second gas sensor.

In one embodiment of the perfusion system, the controller is configured to execute the program stored in the controller to activate delivery of the perfusion fluid when a loss of viability is calculated based on the first concentration of the first gas adjacent the first gas sensor and the second concentration of the second gas adjacent the second gas sensor.

In one embodiment of the perfusion system, the controller is configured to execute the program stored in the controller to deactivate delivery of the perfusion fluid when a calculated viability based on the first concentration of the first gas adjacent the first gas sensor and the second concentration of the second gas adjacent the second gas sensor exceeds a threshold viability value.

In one embodiment of the perfusion system, the controller is configured to execute the program stored in the controller to intermittently activate or deactivate delivery of the perfusion fluid based on the first concentration of the first gas adjacent the first gas sensor and the second concentration of the second gas adjacent the second gas sensor.

In one embodiment, the perfusion system further comprises: a pump in electrical communication with the controller, wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor.

In one embodiment, the perfusion system further comprises: an oxygenator in electrical communication with the controller, wherein the controller is configured to execute the program stored in the controller to control oxygen level in perfusion fluid flowing towards the sample based on the first concentration of the first gas adjacent the first gas sensor and the second concentration of the second gas adjacent the second gas sensor.

In one embodiment, the perfusion system further comprises: a third gas sensor in fluid communication with the outflow conduit, the third gas sensor measuring a concentration of a third gas in perfusion fluid in the outflow conduit, wherein the controller is configured to execute the program stored in the controller to activate or deactivate delivery of the perfusion fluid flowing towards the sample and/or adapt perfusion parameters based on the first concentration of the first gas adjacent the first gas sensor and the second concentration of the second gas adjacent the second gas sensor, and the third concentration of the third gas adjacent the third gas sensor.

In one embodiment of the perfusion system, the controller is configured to execute the program stored in the controller to activate delivery of the perfusion fluid when a loss of viability is calculated based on the first concentration of the first gas adjacent the first gas sensor and the second concentration of the second gas adjacent the second gas sensor, and the third concentration of the third gas adjacent the third gas sensor.

In one embodiment of the perfusion system, the controller is configured to execute the program stored in the controller to deactivate delivery of the perfusion fluid when a calculated viability based on the first concentration of the first gas adjacent the first gas sensor and the second concentration of the second gas adjacent the second gas sensor, and the third concentration of the third gas adjacent the third gas sensor exceeds a threshold viability value.

In one embodiment of the perfusion system, the controller is configured to execute the program stored in the controller to intermittently activate or deactivate delivery of the perfusion fluid based on the first concentration of the first gas adjacent the first gas sensor and the second concentration of the second gas adjacent the second gas sensor, and the third concentration of the third gas adjacent the third gas sensor.

In one embodiment, the perfusion system further comprises: a pump in electrical communication with the controller, wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor, and the third concentration of the third gas adjacent the third gas sensor.

In one embodiment, the perfusion system further comprises: an oxygenator in electrical communication with the controller, wherein the controller is configured to execute the program stored in the controller to control oxygen level in perfusion fluid flowing towards the sample based on electrical signals received from the first gas sensor and the second gas sensor, and the third concentration of the third gas adjacent the third gas sensor.

In one embodiment of the perfusion system, the controller is in electrical communication with a controllable valve in the inflow conduit, and the controller is configured to execute the program stored in the controller to activate or deactivate delivery of the perfusion fluid by moving the controllable valve to an open position in which the perfusion fluid is delivered to the sample or a closed position in which the perfusion fluid is not delivered to the sample.

In one embodiment of the perfusion system, the controller is in electrical communication with a controllable valve in the inflow conduit, and the controller is configured to execute the program stored in the controller to control delivery of the perfusion fluid by moving the controllable valve to a fully open position in which a first amount of the perfusion fluid is delivered to the sample, or an intermediate position in which a second amount of the perfusion fluid less than the first amount of the perfusion fluid is delivered to the sample, or a closed position in which the perfusion fluid is not delivered to the sample.

In another aspect, the present disclosure provides a method for machine perfusion of a tissue sample. The method comprises: (a) providing a tissue sample; (b) delivering a perfusion fluid to the sample via an inflow conduit; (c) measuring a first concentration of a first gas in the perfusion fluid in the inflow conduit; (d) measuring a second concentration of a second gas in the sample; and (e) activating or deactivating delivery of the perfusion fluid to the sample and/or adapting perfusion parameters based on the first concentration of the first gas and the second concentration of the second gas.

In one embodiment of the method, step (e) comprises activating delivery of the perfusion fluid when a loss of viability is calculated based on the first concentration of the first gas adjacent the first gas sensor and the second concentration of the second gas adjacent the second gas sensor.

In one embodiment of the method, step (e) comprises deactivating delivery of the perfusion fluid when a calculated viability based on the first concentration of the first gas adjacent the first gas sensor and the second concentration of the second gas adjacent the second gas sensor exceeds a threshold viability value.

In one embodiment of the method, step (e) comprises intermittently activating or deactivating delivery of the perfusion fluid based on the first concentration of the first gas adjacent the first gas sensor and the second concentration of the second gas adjacent the second gas sensor.

In one embodiment of the method, step (e) comprises adjusting a flow rate of perfusion fluid flowing towards the sample based on the first concentration of the first gas adjacent the first gas sensor and the second concentration of the second gas adjacent the second gas sensor.

In one embodiment of the method, step (e) comprises controlling oxygen level in perfusion fluid flowing towards the sample based on the first concentration of the first gas adjacent the first gas sensor and the second concentration of the second gas adjacent the second gas sensor.

In another aspect, the present disclosure provides a perfusion system for a tissue sample, wherein the system comprises: a perfusion fluid source; an inflow conduit in fluid communication with the perfusion fluid source and the sample, the inflow conduit being configured to deliver perfusion fluid to the sample; an outflow conduit in fluid communication with the sample, the outflow conduit being configured to carry perfusion fluid away from the sample; a first gas sensor in fluid communication with the inflow conduit, the first gas sensor measuring a concentration of a first gas in the perfusion fluid in the inflow conduit; a second gas sensor in fluid communication with the outflow conduit, the second gas sensor measuring a concentration of a second gas in perfusion fluid in the outflow conduit; and a controller in electrical communication with the first gas sensor and the second gas sensor, the controller being configured to execute a program stored in the controller to: (i) receive electrical signals from the first gas sensor and the second gas sensor and to calculate a first concentration of the first gas adjacent the first gas sensor and a second concentration of the second gas adjacent the second gas sensor, and (ii) activate or deactivate delivery of the perfusion fluid flowing towards the sample and/or adapt perfusion parameters based in on the first concentration of the first gas adjacent to the first gas sensor and the second concentration of the second gas adjacent to the second gas sensor.

In one embodiment of the perfusion system, the controller is configured to execute the program stored in the controller to activate delivery of the perfusion fluid when a loss of viability is calculated based on the first concentration of the first gas adjacent the first gas sensor and the second concentration of the second gas adjacent the second gas sensor.

In one embodiment of the perfusion system, the controller is configured to execute the program stored in the controller to deactivate delivery of the perfusion fluid when a calculated viability based on the first concentration of the first gas adjacent the first gas sensor and the second concentration of the second gas adjacent the second gas sensor exceeds a threshold viability value.

In one embodiment of the perfusion system, the controller is configured to execute the program stored in the controller to intermittently activate or deactivate delivery of the perfusion fluid based on the first concentration of the first gas adjacent the first gas sensor and the second concentration of the second gas adjacent the second gas sensor.

In one embodiment, the perfusion system further comprises: a pump in electrical communication with the controller, wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor.

In one embodiment, the perfusion system further comprises: an oxygenator in electrical communication with the controller, wherein the controller is configured to execute the program stored in the controller to control oxygen level in perfusion fluid flowing towards the sample based on the first concentration of the first gas adjacent the first gas sensor and the second concentration of the second gas adjacent the second gas sensor.

In one embodiment, the perfusion system further comprises: a third gas sensor contacting the sample, the third gas sensor measuring a concentration of a third gas in the sample, wherein the controller is configured to execute the program stored in the controller to activate or deactivate delivery of the perfusion fluid flowing towards the sample and/or adapt perfusion parameters based on the first concentration of the first gas adjacent the first gas sensor and the second concentration of the second gas adjacent the second gas sensor, and the third concentration of the third gas adjacent the third gas sensor.

In one embodiment of the perfusion system, the controller is configured to execute the program stored in the controller to activate delivery of the perfusion fluid when a loss of viability is calculated based on the first concentration of the first gas adjacent the first gas sensor and the second concentration of the second gas adjacent the second gas sensor, and the third concentration of the third gas adjacent the third gas sensor.

In one embodiment of the perfusion system, the controller is configured to execute the program stored in the controller to deactivate delivery of the perfusion fluid when a calculated viability based on the first concentration of the first gas adjacent the first gas sensor and the second concentration of the second gas adjacent the second gas sensor, and the third concentration of the third gas adjacent the third gas sensor exceeds a threshold viability value.

In one embodiment of the perfusion system, the controller is configured to execute the program stored in the controller to intermittently activate or deactivate delivery of the perfusion fluid based on the first concentration of the first gas adjacent the first gas sensor and the second concentration of the second gas adjacent the second gas sensor, and the third concentration of the third gas adjacent the third gas sensor.

In one embodiment, the perfusion system further comprises: a pump in electrical communication with the controller, wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor, and the third concentration of the third gas adjacent the third gas sensor.

In one embodiment, the perfusion system further comprises: an oxygenator in electrical communication with the controller, wherein the controller is configured to execute the program stored in the controller to control oxygen level in perfusion fluid flowing towards the sample based on electrical signals received from the first gas sensor and the second gas sensor, and the third concentration of the third gas adjacent the third gas sensor.

In one embodiment of the perfusion system, the controller is in electrical communication with a controllable valve in the inflow conduit, and the controller is configured to execute the program stored in the controller to activate or deactivate delivery of the perfusion fluid by moving the controllable valve to an open position in which the perfusion fluid is delivered to the sample or a closed position in which the perfusion fluid is not delivered to the sample. In one embodiment of the perfusion system, the controller is in electrical communication with a controllable valve in the inflow conduit, and the controller is configured to execute the program stored in the controller to control delivery of the perfusion fluid by moving the controllable valve to a fully open position in which a first amount of the perfusion fluid is delivered to the sample, or an intermediate position in which a second amount of the perfusion fluid less than the first amount of the perfusion fluid is delivered to the sample, or a closed position in which the perfusion fluid is not delivered to the sample.

In another aspect, the present disclosure provides a method for machine perfusion of a tissue sample. The method comprises: (a) providing a tissue sample; (b) delivering a perfusion fluid to the sample via an inflow conduit; (c) measuring a first concentration of a first gas in the perfusion fluid in the inflow conduit; (d) measuring a second concentration of a second gas in an outflow conduit; and (e) activating or deactivating delivery of the perfusion fluid to the sample and/or adapting perfusion parameters based on the first concentration of the first gas and the second concentration of the second gas.

In one embodiment of the method, step (e) comprises activating delivery of the perfusion fluid when a loss of viability is calculated based on the first concentration of the first gas adjacent the first gas sensor and the second concentration of the second gas adjacent the second gas sensor.

In one embodiment of the method, step (e) comprises deactivating delivery of the perfusion fluid when a calculated viability based on the first concentration of the first gas adjacent the first gas sensor and the second concentration of the second gas adjacent the second gas sensor exceeds a threshold viability value.

In one embodiment of the method, step (e) comprises intermittently activating or deactivating delivery of the perfusion fluid based on the first concentration of the first gas adjacent the first gas sensor and the second concentration of the second gas adjacent the second gas sensor.

In one embodiment of the method, step (e) comprises adjusting a flow rate of perfusion fluid flowing towards the sample based on the first concentration of the first gas adjacent the first gas sensor and the second concentration of the second gas adjacent the second gas sensor.

In one embodiment of the method, step (e) comprises controlling oxygen level in perfusion fluid flowing towards the sample based on the first concentration of the first gas adjacent the first gas sensor and the second concentration of the second gas adjacent the second gas sensor.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram of the perfusion system, along with the fasciocutaneous flap and the oxygen monitoring devices, according to aspects of the present disclosure.

FIG. 2A is a side view of the film of the perfusion system of FIG. 1 over a perfusion fluid source.

FIG. 2B is a side view of the film of the perfusion system of FIG. 1 over a tissue sample.

FIG. 3 is a perspective view of the sensor head of the perfusion system of FIG. 1.

FIG. 4 is a flow chart of the method for machine perfusion of a tissue sample, according to aspects of the present disclosure.

FIG. 5 is a plot of the monitoring of the transcutaneous (TcPO₂ orange curve, right Y axis) and circulating inflow (PO₂, blue curve, left Y axis) oxygen values during extended intermittent perfusion of a fasciocutaneous flap. Inflow values were compared to intermittent sampling and gas analysis in the perfusate. The inset depicts the time delay between the rise in oxygen of the perfusate and the skin.

FIG. 6 is a plot of the measurements in a 3-device system providing transcutaneous (orange curve, right Y axis), circulating inflow (dark blue, left Y axis) and outflow (light blue, left Y axis) oxygen values.

FIG. 7 is a plot of the statistically significant correlation between continuous PO₂ values provided by the flowcell device and by a Siemens Rapidpoint 500 blood gas analyzer. The 95% confidence interval is represented in dashed lines.

FIG. 8A is the maximum PO₂ and TcPO₂ values during ex-vivo perfusion of a fasciocutaneous flap.

FIG. 8B is the minimum PO₂ and TePO₂ values during ex-vivo perfusion of a fasciocutaneous flap.

FIG. 8C is the change (Δ=Max−Min) in PO₂ and TcPO₂ values during ex-vivo perfusion of a fasciocutaneous flap.

FIG. 8D is the delay between circulating inflow PO₂ and skin TcPO₂ showing a similar trend towards disconnection between inflow and skin values. The vascular resistance is also plotted, showing a strong correlation with the delay.

Like reference numerals will be used to refer to like parts from Figure to Figure in the following description of the drawings.

### DETAILED DESCRIPTION

The method of assessing perfused tissue viability described herein involves continuous measurement of partial oxygen pressure (pO₂) at multiple locations, including but not limited to the oxygen-rich (inflow) and oxygen-depleted (outflow) perfusate, at depths in the tissue, and the surface of the perfused tissue, and correlation of the measurement results. One embodiment employs: (a) a tissue perfusion system that circulates oxygenated perfusate through tissue via vascular connection and drainage; (b) oxygen-sensing flow-cell within or part of the system tubing and a needle, microneedle, or skin patch type materials placed into/onto the perfused tissue surface, respectively; (c) programmable, electronic readout sensors for the oxygen-sensing materials for collecting the signal; (d) an algorithm loaded onto a computer or mobile device that controls the readout devices that may log, process and report the signal output from multiple devices in realtime in the form of pO₂ values; and (e) an algorithm loaded onto a computer or mobile device that controls the perfusion system pump and pressure sensor for adapting the perfusion parameters depending on the oxygen measurement levels.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined by the claims. The features illustrated or described in connection with one exemplary embodiment may be combined with the features of other embodiments. Such modifications and variations are intended to be included within the scope of the present invention.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

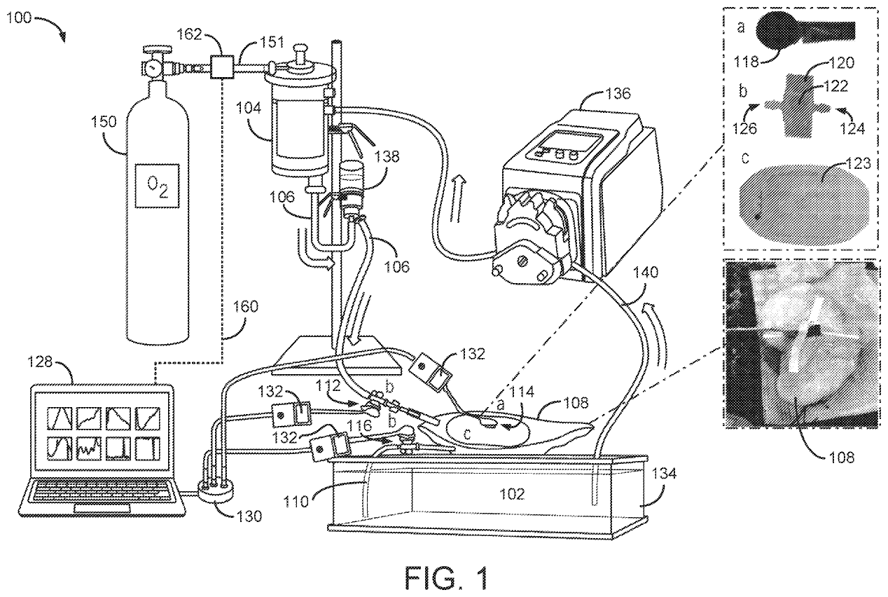

FIG. 1 shows an example embodiment of the perfusion system 100 of the disclosure. The perfusion system 100 includes a perfusion fluid source 102. In a non-limiting example, the perfusion fluid 102 is an acellular crystalloid solution. The system 100 includes a membrane oxygenator 104 allowing for oxygenation of the circulating perfusion fluid 102. The membrane oxygen may receive its oxygen supply 150 via tube 151. The system 100 further includes an inflow conduit 106 in fluid communication with the perfusion fluid source 102 and a tissue sample 108 via an artery. In a non-limiting example, the tissue sample 108 is a fasciocutaneous flap. In a non-limiting example, the inflow conduit 106 is a tube. For example, the tube may be, but is not limited to, silicone tubing or polyvinyl chloride (PVC) tubing. The system 100 further includes an outflow conduit 110 in fluid communication with the sample, the outflow conduit 110 being configured to carry perfusion fluid away from the sample. The outflow conduit 110 may be the same material as the inflow tubing 106. The perfusion fluid carried away from the tissue sample 108 by a vein via the outflow conduit 110 has perfused through the sample 108. The system 100 further includes a first gas sensor 112 in fluid communication with the inflow conduit 106, and a second gas sensor 114 contacting the sample 108. The first gas sensor 112 measures a concentration of a gas in the perfusion fluid 102, while the second gas sensor 114 measures a concentration of a gas in the sample 108.

In an alternative embodiment, the perfusion system 100 includes a third gas sensor 116 as shown in FIG. 1. The third gas sensor 116 may be identical to the first gas sensor 112 as previously described. In a non-limiting example, the third gas sensor is in fluid communication with the outflow conduit 110, configured to measure a concentration of a gas in perfusion fluid in the outflow conduit 110.

In a non-limiting example, the perfusion fluid may be a Composition of Steen+ Solution—Massachusetts General Hospital, Center for Engineering in Medicine and Surgery, Department of Surgery, Harvard Medical School, Boston MA 02114 USA. The Steen+ solution contains, in a de-ionized water basis:
NaCl: 86 mmol/L
KCl: 4.6 mmol/L
CaCl₂·2H₂0: 1.5 mmol/L
NaH₂PO₄: 1.2 mmol/L
NaHCO₃: 16 mmol/L
MgCl₂·6H₂O: 1.2 mmol/L
D-Glucose: 22 mmol/L
Polyethylene Glycol (PEG) 35 Kda: 5 g/L
Bovine Serum Albumin: 15 g/L
Insulin: 200 UI/L
Hydrocortisone: 10 mg/L
Heparin: 200 UI/L
Piperacillin-Tazobactam: 2.25 G/L
Vancomycin: 1.5 G/L

In a non-limiting example, the first gas sensor 112 and optionally the third gas sensor 116 include a sensor head 118 and flow cell 120 containing a film 122. In one embodiment, the flow cell 120 of the first gas sensor is in communication with the inflow conduit 106, while the flow cell 120 of the third gas sensor is in communication with the outflow conduit 110, and the film 122 contained within the flow cell 120 is in contact with the perfusion fluid 102. In a non-limiting example, the flowcell can be incorporated into the perfusion system by cutting the inflow tubing and plugging the flowcell inside the tubings, restoring the continuity of the tubing. The flowcell is designed to have an internal diameter close to one of the silicone tubings. Likewise, the outflow sensor can be similarly placed. In a non-limiting embodiment, the flow cell 120 includes a first opening 124 configured to allow the perfusion fluid 102 to enter the flow cell 120 and a second opening 126 configured to allow the perfusion fluid 102 to exit the flow cell 120.

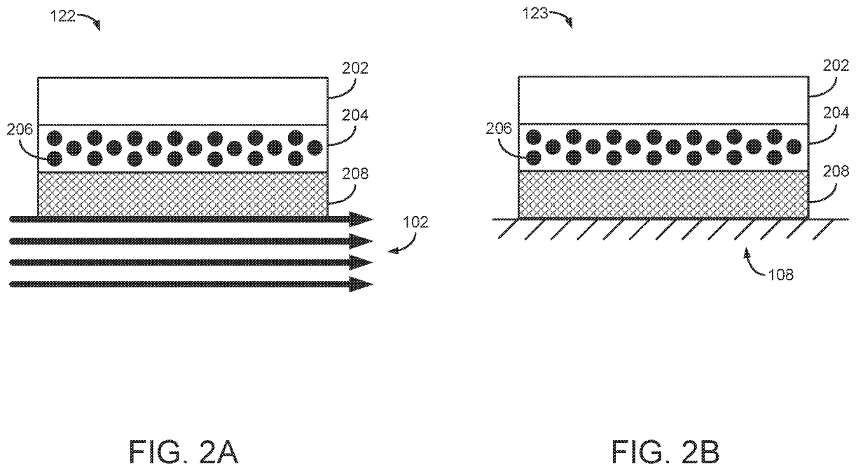

The film 122 or 123 is configured to undergo a change in response to pO₂. The film 122 or 123 can have a light-based, photoluminescent oxygen sensing formulation that can have emission properties that can provide tissue pO₂. The film 122 or 123 can include an oxygen-sensing polymer or polymer containing an oxygen-sensing molecule. As shown in FIG. 2A, the film 122 or 123 may include three layers: (i) a transparent membrane 202 forming an outer layer of the film, (ii) a layer of polymeric material 204 embedded with an oxygen-sensing lumiphore 206 adjacent to the transparent membrane 202, and (iii) a scattering layer 208 (such as a polymeric film pigmented with white particles) in contact with the layer of the polymeric material 204 and the perfusion fluid 102.

In a non-limiting example, the oxygen-sensing lumiphore 206 can be a metalloporphyrin can emit red phosphorescence when excited by blue light, and the phosphorescence intensity and lifetime can be inversely proportional to pO₂. A reference sensor in the form of a green-emitting dye can also be incorporated into the sensor film 122 or 123 to serve as a reference standard for precise pO₂ measurements.

In a non-limiting example, the polymer may be, but is not limited to poly(propyl methacrylate) (PMMA) or polydimethylsiloxane (PDMS). For instance, silicones such as PDMS can be extremely gas-permeable and can allow rapid readout of tissue oxygen dynamics.

In some embodiments, porphyrin-based, oxygen sensing molecules 206 embedded in the polymeric material 204 are designed to provide extremely high sensitivity and accuracy for the measurement of tissue oxygenation. Porphyrin-based, oxygen sensing molecules 206 can be built via a modular synthetic pathway that enables the tailoring of both the oxygen sensing molecules' oxygen sensitivity range and the oxygen sensing molecules' compatibility with the matrix material the oxygen sensing molecules can be embedded in. The matrix material can further be configured to tailor the oxygen sensing molecules' oxygen sensitivity range. In one embodiment the change in the film 122 or 123 includes a change in the phosphorescence. For example, the oxygen sensing molecules 206 can be specifically designed to feature bright, red phosphorescence emission, with a visual response to changes in oxygenation level that can be seen under ambient light. These properties simplify the collection and interpretation of their oxygen-dependent emission, enabling the analysis to be performed with simple and inexpensive equipment.

In one embodiment, the oxygen-sensing lumiphore 206 comprises a phosphorescent meso-unsubstituted porphyrin having the Formula (I):

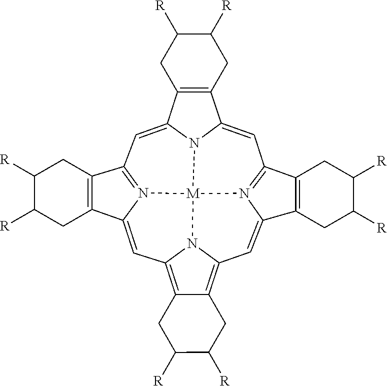

wherein M is a metal, wherein each R is independently an atom or a group of atoms, and wherein at least one R is —OR¹, wherein R¹ is an atom or a group of atoms.

In the porphyrin of Formula (I), R¹ may be selected from the group consisting of hydrogen, substituted or unsubstituted alkyl, substituted or unsubstituted alkyl carbonyl, substituted or unsubstituted alkenyl, substituted or unsubstituted alkynyl, substituted or unsubstituted cycloalkyl, substituted or unsubstituted heterocycloalkyl, substituted or unsubstituted aryl, heteroaryl, halo, cyano, and nitro. In one example of the porphyrin of Formula (I), R¹ is hydrogen. In another example of the porphyrin of Formula (I), R¹ is alkynyl, such as 2-propynyl (propargyl). In yet another example of the porphyrin of Formula (I), R¹ is alkyl carbonyl, such as 2,2-dimethylpropanoyl (also known as trimethylacetyl or pivaloyl). In the porphyrin of Formula (I), a plurality of R can be —OR¹, and optionally, every R can be —OR¹.

In one example of the porphyrin of Formula (I), R¹ includes a triazolyl group. The triazolyl group may be bonded to O via an alkyl chain. In one example of the porphyrin of Formula (I), R¹ includes an alkylglutamate group. R¹ may terminate in a pair of alkylglutamate groups. In another example of the porphyrin of Formula (I), R¹ includes a triazolyl group, and R¹ terminates in a pair of ethylglutamate groups, and every R is —OR¹. In one example of the porphyrin of Formula (I), the metal is platinum or palladium.

The porphyrin of Formula (I) may be an oxygen-sensitive phosphor whose emission intensity is dependent on oxygen partial pressure. In one example of the porphyrin of Formula (I), the porphyrin can be excited when illuminated at a first wavelength in a range of 350-600 nanometers, followed by emission of phosphorescence at a second wavelength in a range of 600-700 nanometers. The first wavelength can be 532 nanometers, and the second wavelength can be 644 nanometers. The first wavelength can also be 546 nanometers and the second wavelength can be 674 nanometers.

In another embodiment, the oxygen-sensing lumiphore 206 comprises a phosphorescent meso-unsubstituted porphyrin having the Formula (II):

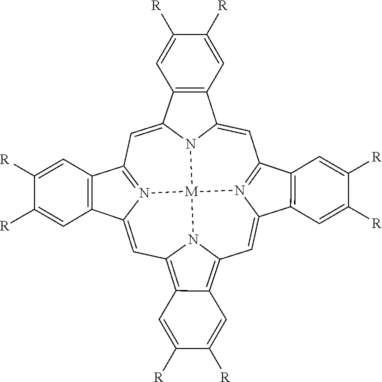

wherein M is a metal, wherein each R is independently an atom or a group of atoms, and wherein at least one R is —OR¹, wherein R¹ is an atom or a group of atoms.

In the porphyrin of Formula (II), R¹ may be selected from the group consisting of hydrogen, substituted or unsubstituted alkyl, substituted or unsubstituted alkyl carbonyl, substituted or unsubstituted alkenyl, substituted or unsubstituted alkynyl, substituted or unsubstituted cycloalkyl, substituted or unsubstituted heterocycloalkyl, substituted or unsubstituted aryl, heteroaryl, halo, cyano, and nitro. In one example of the porphyrin of Formula (II), R¹ is hydrogen. In another example of the porphyrin of Formula (II), R¹ is alkynyl, such as 2-propynyl (propargyl). In yet another example of the porphyrin of Formula (II), R¹ is alkyl carbonyl, such as 2,2-dimethylpropanoyl (also known as trimethylacetyl or pivaloyl). In the porphyrin of Formula (II), a plurality of R can be —OR¹, and optionally, every R can be —OR¹.

In one example of the porphyrin of Formula (II), R¹ includes a triazolyl group. The triazolyl group may be bonded to O via an alkyl chain. In one example of the porphyrin of Formula (II), R¹ includes an alkylglutamate group. R¹ may terminate in a pair of alkylglutamate groups. In another example of the porphyrin of Formula (II), R¹ includes a triazolyl group, and R¹ terminates in a pair of ethylglutamate groups, and every R is —OR¹. In one example of the porphyrin of Formula (II), the metal is platinum or palladium.

The porphyrin of Formula (II) may be an oxygen-sensitive phosphor whose emission intensity is dependent on oxygen partial pressure. In one example of the porphyrin of Formula (II), the porphyrin can be excited when illuminated at a first wavelength in a range of 350-650 nanometers, followed by emission of phosphorescence at a second wavelength in a range of 700-800 nanometers. The first wavelength can be 594 nanometers, and the second wavelength can be 740 nanometers. The first wavelength can be 605 nanometers, and the second wavelength can be 770 nanometers. The first wavelength can also be 600-615 nanometers and the second wavelength can be 760-800 nanometers.

In a non-limiting embodiment, the second gas sensor 114 includes the photoluminescent oxygen-sensitive probe comprising a polymeric material impregnated with a porphyrin as disclosed for the first and third gas sensors 112, 116 above. The photoluminescent oxygen-sensitive probe is a formulation having an emission that provides tissue pO₂. For example, the second gas sensor 114 is a skin sensor placed within or on a fasciocutaneous flap after its harvesting, and before starting the perfusion. In one example, the second gas sensor 114 is an oxygen sensing needle that can be placed within the center tissue 108. In another example, a sensor film can be covered by a transparent adhesive dressing (for example Tegaderm™, 3M, St Paul, MN). In a non-limiting example, the film 123 as previously described above is in contact with the surface of the tissue sample 108. As shown in FIG. 2B, the scattering layer 208 is in contact with polymeric material 204 and a surface of the sample 108. The second gas sensor 114 provides tissue pO₂.

In some embodiments, the sensor head 118 can interface with a porphyrin described in U.S. Patent Application Publication No. 2016/0159842, which is incorporated herein by reference. For example, the porphyrin may be an oxygen-sensitive phosphor whose emission intensity is dependent on oxygen partial pressure.

In a non-limiting example, the first, second, and third gas sensors 112, 114, 116 can each incorporate a probe head 118 placed in contact with a film 122 or 123 containing a light-based oxygen sensing formulation that can have an emission that can provide tissue pO₂, emission sources, and detectors that can be mounted on the gas sensors 112, 114, 116. The sensor heads may be those previously described in PCT Patent Application Publication No. WO 2017/197385, which is herein incorporated by reference.

In a non-limiting example, the first, second, and third gas sensor 112, 114, 116 can have a light-based, photoluminescent oxygen sensing formulation in the film 122 or 123, that can have an emission that can provide tissue pO₂. The sensor heads 118 can interface with an oxygen-sensing film 122 or 123 or polymer containing an oxygen-sensing molecule. In the first and/or third gas sensor 112, 116, the sensor head 118 can be positioned on the flow cell 120 to contact an inner surface of the inflow conduit 106 and/or outflow conduit 110= and thus come into direct contact with the perfusion fluid. The second gas sensor 114 can be positioned to be in direct contact with the sample tissue 108. The probe heads 118 can be in the form of a circular pad that interfaces with a film containing oxygen sensing molecules. The film can also contain other sensors such as reference sensors. The reference sensors can provide a baseline for oxygenation measurement or a reference for calibrating the oxygen sensor. In another embodiment, the probe head 118 can be integrated in a single package with the flow cell 120 and sensing film 122.

The second gas sensor 114 can have an interface mechanism that can be configured to place the probe head 118 in contact with the film 123 sample tissue 108. In some embodiments, the interface mechanism can provide contact between the second gas sensor 114 and a tissue 108 using an adhesive that adheres an interface surface of the second gas sensor 114 to the tissue 108. The interface mechanism in the form of an adhesive can directly and reversibly adhere the second gas sensor 114 to the tissue 108. In other embodiments, the interface mechanism can be a strap, a band, an elastic element, a pocket, or any other suitable interface mechanism capable of placing the second gas sensor 114 in contact with a tissue of a patient. In a non-limiting example, a seal may be utilized between the film 122 and the tissue 108.

In one embodiment, the first, second, and third gas sensors 112, 114, 116 each include a sensor head 118. The sensor head 118 may include a photon source configured to direct photons at the photoluminescent oxygen-sensitive film 122 or 123. The sensor head 118 may include a photodetector configured to detect light emitted from the photoluminescent oxygen-sensitive film 122 or 123 when the photon source directs photons at the photoluminescent oxygen-sensitive film 122. The sensor head 118 can also include a controller in electrical communication with the photon source and the photodetector, the controller being configured to execute a program stored in the controller to calculate a concentration of oxygen adjacent to the photoluminescent oxygen-sensitive film 122 or 123 form an electrical signal received from the photodetector.

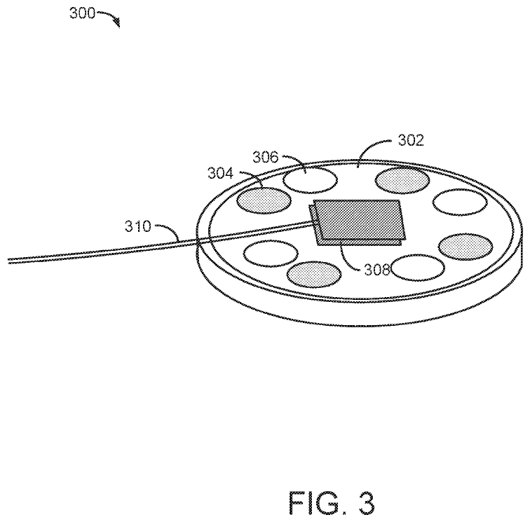

FIG. 3 is a non-limiting example of a sensor head 118. It shows a circuit board 302 containing the photon sources 304 and the detectors 306 that can be attached to the sensor head 300. In some embodiments, the circuit board 302 can be attached to the sensor head 300 on an opposite side of the sensor head relative to the photon sources 304 and detectors 306. In some embodiments, the circuit board 302 can be linked to the sensor heads 300 via optical fibers (not shown). The circuit board 302 can be flexible and can have a substrate, wherein the emission sources 304 and the detectors 306 can be embedded in or on the circuit board 302 substrate.

The emission sources 304 can be positioned in or on the substrate such that each photon source 304 is positioned to emit photons directed at the film 122 or 123. In an exemplary, non-limiting embodiment, the photon sources 304 are positioned at four radial positions around the circuit board 302 such that the photon sources 304 can direct photons to the film 122 or 123. In some embodiments, the photon sources 304 can be a blue light-emitting diode. In other embodiments, the photon sources 304 can be a green, yellow, or orange light-emitting diode. In other embodiments, the photon sources 304 can be optical fibers that deliver specific colors of light.

The detectors 306 can be positioned in or on the substrate such that each of the detectors 306 is positioned to detect or receive photons emitted from the film 122 or 123. In an exemplary, non-limiting embodiment, the detectors 306 are positioned at four radial positions around the circuit board 302 such that the detectors 306 can detect or receive photons emitted from the film 122. The detectors 306 on the circuit board 302 can comprise one or more photodetectors. In one non-limiting embodiment, the photodetectors can be configured to be sensitive to different wavelengths of light. In some embodiments, the detectors 306 can be a photodiode. The one or more green photodetectors can be green photodiodes, and the one or more red photodetectors can be photodiodes. In other embodiments, the detectors 306 can be a charge-coupled device (CCD). In other embodiments, the detectors 306 can be optical fibers that couple emitted light to photodetectors, including photodiodes and charge-coupled devices (CCDs). In other embodiments, the photodetectors can detect both green and red emission.

The circuit board 302 can also comprise a controller 308 that can be in electrical communication with the photon sources 304 and the detectors 306. The controller 308 can be a microcontroller, or system-on-a-chip, and can comprise a memory which can be a non-transitory memory that can store executable programs on the controller 308. In some embodiments, the controller 308 can store an oxygen calculation program that can calculate a level of oxygen adjacent the sensor head 118, 300 from one or more electrical signals received from the detectors 306. The circuit board 302 can also include an output 310, which can be a wire bundle. The output 310 can connect to an external interface 128 in FIG. 1 which can be used for at least one of displaying, storing, and analyzing the results of the executable program. In one example, the output 310 can connect to the external interface 128 via a universal serial bus (USB) hub 130. In other embodiments, the controller 308 can be configured to have a wireless output, the wireless output can perform wireless communication. Non-limiting examples of wireless communication that can be incorporated are Wi-Fi, Bluetooth®, near-field communication, cellular network, radiofrequency, etc. The circuit board 302 can comprise an on-board power source (not shown), for example a battery, that can provide power to the circuit board 302 such that the photon sources 304, the detectors 306, and the controller 308 can be powered. In other embodiments, the circuit board 302 can comprise an external power source (not shown), for example electrical connection to grid power, that can provide power to the circuit board 302 such that the photon sources 304, the detectors 306, and the controller 308 can be powered.

Since optical first, second, and third gas sensors 112, 114, 116 can directly measure pO₂, it does not require perfusion with oxygen carriers such as red blood cells or whole blood. The sensing components can be safely retained within the sensor head 118, 300, which can be non-invasive, and as such, exogenous dyes, injectable agents, and needles may not be required. The gas sensors 112, 114, 116 can require low preparation time, and a readout can be essentially instantaneous.

Now that the components of the perfusion system 100 components have been described in detail, the function of the components can be understood. In some embodiments, the sensor head 118 can be placed in contact with the film 122 in the flow cell 120 or with the film 123 on the tissue sample 108 (FIGS. 2A-2B). In some embodiments, films 122 and 123 can include an oxygen-sensing polymer that can be in direct contact with the perfusion fluid 102 or tissue sample 108 of the patient. Direct contact between the sensor head 118 and the film 123 on the tissue of the patient can allow for the sensor head 118 to provide tissue pO₂ via the photoluminescent oxygen sensing formulation of the film 123. The photoluminescent oxygen sensing formulation of the film 123 can have an emission that can be indicative of the tissue pO₂.

The photoluminescent oxygen sensing formulation of the films 122 and 123 can emit red phosphorescence when excited by blue light from one or more of the photon sources 304, with a phosphorescence intensity detected by one or more of the detectors 306. The phosphorescence intensity can be inversely proportional to pO₂ of the tissue of the patient. A reference sensor, which can be in the form of a green-emitting dye, can also be loaded into the films 122 and 123 and can serve as a reference standard for precise pO₂ measurements. In a non-limiting embodiment, the phosphorescence lifetime can be also inversely proportional to pO₂ of the tissue of the patient, and alternatively or additionally measured and analyzed to provide precise pO₂ measurements.

The circuit board 302 can be flexible and can be attached to a surface of the sensor heads 118 via an oxygen-impermeable membrane. The circuit board 302 can contain photon sources 304 and detectors 306. The photon sources 304 can be one or more blue light emitting diodes (LEDs), and the detectors 306 include one or more photodetectors in the form of green sensitive photodiode detectors and one or more red photodetectors in the form of red sensitive photodiode detectors. In some embodiments, the detectors can be a photodiode, photomultiplier tube, avalanche photodiode, charge-coupled device (CCD), complementary metal-oxide semiconductor (CMOS) device, or combination of similar photodetectors.

The oxygen-dependent change in red phosphorescence intensity, can be captured by the red photodetectors in the form of red sensitive photodiode detectors and referenced against the green emission captured by the green photodetectors in the form of green sensitive photodiode detectors using analog circuitry of the controller 308 to provide pre-calibrated, robust transcutaneous oxygen tension measurements of the sample 108.

The oxygen-dependent change in phosphorescence lifetime can be captured by the photodetectors in the form of photodiode detectors. LED illumination can be modulated (e.g., by a sinusoidal wave), causing a (e.g., sinusoidally) modulated phosphorescence emission from the oxygen sensors whose time delay with respect to the excitation light can be measured to calculate phosphorescence lifetime, and thus pO₂.

The analog or digital circuitry of the controller 308 can provide pre-calibrated, robust oxygen tension measurements of the tissue of the patient. In some embodiments, the oxygen tension measurements can be analyzed and reported by utilizing molecules (e.g., fluorophore probes) whose emission properties are insensitive to oxygen along with molecules (e.g., phosphor probes) whose emission properties are influenced by molecular oxygen concentration. The molecules whose emission properties are insensitive to oxygen can be the reference sensor, and the molecules whose emission properties are influenced by molecular oxygen concentration can be the phosphor probes. Emission from the fluorophore probes and phosphor probes can be used to measure oxygen tension in biological systems reversibly with high fidelity. The fluorophore probes and phosphor probes can be calibrated so that a spectral ratio between fluorophore and phosphor emission correlates with oxygen concentration of the tissue sample 108. The calibration can be used to read out a map of oxygen concentration in the tissue of the patient. The calibration can also be used to read out an average oxygen concentration of the area covered by the first, second, or third gas sensor 112, 114, 116. The fluorophore probes and phosphor probes can be calibrated so that the lifetime of the fluorophore and phosphor emission can be analyzed to provide the oxygen concentration of the tissue sample 108. It is also possible to utilize molecules such as dyes whose light absorption properties (such as, absorption wavelength or absorption cross-section) can be modulated by the presence of analytes such as oxygen for light absorption-based colorimetric oxygen measurements.

In one embodiment, the analog or digital circuitry of the controller 308 can provide pre-calibrated, robust transcutaneous oxygen tension measurements and analysis of the tissue sample 108 using the Stern-Volmer relationship. The Stern-Volmer relationship can be used to characterize the oxygenation of the fluid 102 or sample 108 based on the photoluminescent oxygen sensing formulation of the film 122 or 123, respectively, that can emit red phosphorescence when excited by blue light from the at least one photon source 304, with a phosphorescence intensity and/or lifetime detected by the at least one detector 306 can be inversely proportional to pO₂ of the tissue of the patient. A dynamic (collisional) quenching by oxygen is a photophysical (rather than a photochemical) process. It is fully reversible, does not alter the optical probe, and thus has no effect on its absorption spectrum. Rather, it leads to a change in luminescence intensity and luminescence lifetime. The relationship between intensity (or decay time) and the concentration of oxygen ([O₂]) is reflected by the Stern-Volmer equation which, in its most simple form, reads as:

$$F = \frac{{F}_{0}}{1 + {K}_{SV} \cdot [ {O}_{2} ]} ; τ = \frac{{τ}_{0}}{1 + {K}_{SV} \cdot [ {O}_{2} ]}$$

where F₀ and F, respectively, are the luminescence intensities of a probe in the absence and presence of oxygen, to and r, respectively, are the luminescence lifetimes of a probe in the absence and presence of oxygen, K_{SV} is the Stern-Volmer constant which is a function of the lifetime of the probe and its environment (polymeric matrix, solvent, etc.), and [O₂] is the concentration of oxygen in the sample. The term [O₂] (a concentration) may be replaced by pO₂, the partial pressure of oxygen.

In some embodiments, there can be a linear relationship between F₀/F (or τ₀/τ) and oxygen concentration. Stern-Volmer plots (SVPs) can be established by measurement of either luminescence intensity or lifetime. However, luminescence intensity data can be adversely affected by poor stability of the light source, variations in the efficiency of the transmission optics, drifts in detector sensitivity, leaching and photodecomposition of probes, inhomogeneous probe distribution, background luminescence and stray light. In order to correct for these effects, a reference sensor (e.g., an inert reference fluorophore emitting at a different wavelength) can be used. Alternatively, to correct for these effects, phosphorescence lifetime can be measured which is not adversely affected by such factors.

In some embodiments, the controller 308 can analyze the lifetime and intensity of the phosphorescence emission to determine the oxygen concentration of the sample 108. In other embodiments, the first, second, or third gas sensor 112, 114, 116 can communicate emission results acquired from the at least one detector 306 externally to be analyzed by an external device 128.

In some embodiments, the first and second sensor head 118 can also comprise a display 132. The display 132 can be configured to indicate the oxygenation of the tissue of the patient as determined by the first, second, or third gas sensors 112, 114, 116. In some embodiments, the display can be attached to the sensor head 118, while in other embodiments, the display can be positioned externally, such as in the external interface 128.

In another embodiment the second gas sensor comprises an oxygen-sensitive probe configured for insertion into the sample 108. In one example, the second gas sensor may include a transcutaneous sensor and a separate oxygen-sensing needle, as described herein. For example, the oxygen-sensitive probe may be an oxygen-sensing needle and placed in the center of the tissue sample 108 to perform subdermal, intramuscular, or intra organ pO₂ measurements.

In another embodiment, the perfusion system 100 comprises an oxygenator 104 in fluid communication with the perfusion fluid source 102. For example, the oxygenator 104 is connected to the inflow conduit 106. Further, the oxygenator 104 controls oxygen level in perfusion fluid flowing towards the sample 108. In a non-limiting example, a controller, such as the external interface device 128 in electrical communication with the first gas sensor, the second gas sensor, and/or third gas sensor is configured to execute a program stored in the controller to receive electrical signals from the first gas sensor, the second gas sensor, and/or the third gas sensor and to calculate a concentration of oxygen adjacent the first gas sensor, the second gas sensor, and/or the third gas sensor. In another non-limiting example, the controller such as the external interface device 128 in electrical communication with the first gas sensor, the second gas sensor, and/or third gas sensor is configured to execute a program stored in the controller in response to the information from the first, second, and/or third gas sensors to change or modulate the perfusion of the tissue sample. In another non-limiting aspect, the controller 128 is in electrical communication 160 with a valve 162 to change or modulate the perfusion of the tissue sample.

The perfusion system 100 may further include a pump 136 for circulating the perfusion fluid 102 through the oxygenator 104, inflow conduit 106, tissue sample 108, the outflow conduit 110, a reservoir 134, and a recirculating conduit 140. The perfusion system 100 may further include a bubble trap 138 positioned in line between the oxygenator 104 and the first gas sensor 112. The bubble trap 138 is configured to remove bubbles in the perfusion fluid 102 formed as a result of flowing through the oxygenator 104.

In another embodiment, the perfusion system 100 comprises a heat exchanger (not shown) for adjusting the temperature of the perfusion fluid.

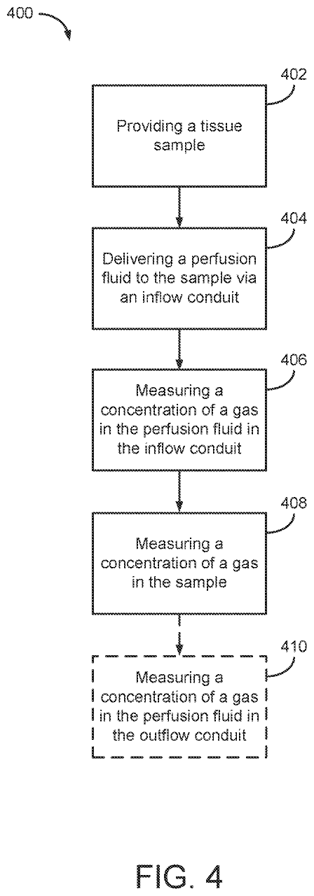

The present disclosure also provides a method 400 for machine perfusion of a tissue sample, as shown in the flowchart of FIG. 4. The method 400 includes step 402 of providing a tissue sample. The tissue sample may be a vascular composite allograft. For example, the vascular composite allograft is at least a portion of a limb, face, larynx, trachea, abdominal wall, genitourinary tissue, uterine tissue, or solid organ, or any combination thereof. In another embodiment, the tissue sample is a donor vascular composite allograft for vascular composite allograft transplantation. In one example, the tissue sample is obtained from a human, a primate, or a pig. In another example, the tissue sample is a fasciocutaneous flap. At step 404, a perfusion fluid is delivered to the sample via an inflow conduit. At step 406, a concentration of a gas in the perfusion fluid in the inflow conduit is measured. In a non-limiting example, the measurement is performed using a first gas sensor, such as the first gas sensor 112 as described above. At step 408, a concentration of gas is measured in the sample. In a non-limiting example, the measurement is performed using a second gas sensor, such as the second gas sensor 114 as described above. Alternatively, the measurement is performed using an oxygen-sensing needle as previously described.

In one example, the first gas sensor, second gas sensor, third gas sensor, or all or a combination of sensors comprise an oxygen-sensitive probe. In another example the first gas sensor, second gas sensor, third gas sensor, all or a combination of sensors comprise a film configured to undergo a change in response to pO₂.

In one example, step 406 further comprises sensing the concentration of gas in the perfusion fluid in the inflow conduit with a first gas sensor and calculating, in a controller, a concentration of oxygen adjacent to the first gas sensor from an electrical signal transmitted to the controller from the first gas sensor. Likewise, step 408 further comprises measuring the concentration of the gas in the sample with the second gas sensor, and calculating, in the controller, a concentration of oxygen adjacent to the second gas sensor from an electrical signal transmitted to the controller from the second gas sensor.

Optionally, the method 400 may include step 410 comprising sensing the concentration of gas in the perfusion fluid in the outflow conduit with a third gas sensor and calculating, in a controller, a concentration of oxygen adjacent to the third gas sensor from an electrical signal transmitted to the controller from the third gas sensor.

In an alternative embodiment, the method may comprise steps 402-406 and 410, omitting step 408.

In a non-limiting example, a report is generated, using the controller, of oxygen perfusion in the sample from measurements from the first, second, and/or third gas sensors.

In a non-limiting example, the method comprises controlling an oxygen level in the perfusion fluid flowing towards the sample based on electrical signals received from the first gas sensor, the second gas sensor, and/or the third gas sensor.

In a non-limiting example, the method comprises activating an alarm when a gas level drops below a defined threshold based on electrical signals received from the first gas sensor, second gas sensor, and/or third gas sensor or any calculation on the data collected from the sensors. In a non-limiting example, the method comprises adapting the perfusion parameters to the data collected from the sensors, by sending a signal to the perfusion system's pump or altering the perfusion system's oxygenation parameters

In one embodiment, the tissue sample is perfused intermittently, i.e., generating perfusion cycles alternating between baseline and high oxygen partial pressure in the perfusate.

In one embodiment of this invention, a 3-device measurement (including measurements from the first, second, and third gas sensors) connected to intermittently perfused sample, provides, simultaneous, continuous, real-time inflow and outflow pO₂ values in the perfusate as well as transcutaneous pO₂ values on the perfused tissue surface. Intermittent perfusion allows the observation of the response of the tissue to changes in oxygenation which is dependent on the state of the capillary bed of the sample, and therefore the tissue viability (e.g., correlation or decorrelation between the sensor measurements). When the tissue is viable, transcutaneous pO₂ follows the trend of the inflow measurement matching the pO₂ increase/decrease according to the operator-induced perfusate oxygenation cycles (oxygenator on/off, oxygen levels varied periodically, or oxygen levels varied through a programmable means). The outflow measurement may show a depletion in pO₂ as well as the same oxygenation cycle-dependent trend. When the tissue becomes non-viable, no more change is observed in tissue pO₂ over time (decorrelation) and the outflow measurement shows an increase in pO₂ due to the tissue no longer consuming oxygen. Other quantities which reflect tissue non-viability can be the difference in oxygenation between inflow and sample, inflow and outflow. Alternatively, the time difference in the increase/decrease in oxygenation of the sample with respect to the perfusate at the inflow, or the sample with respect to the outflow, or the inflow with respect to the outflow, shows a decrease indicative of the tissue becoming non-viable.

### EXAMPLE

Described herein are examples to demonstrate and further illustrate certain embodiments and aspects for the combination of intermittent perfusion and continuous measurement of pO₂ of the perfusate entering/exiting the tissue and the pO₂ of the tissue itself (transcutaneous, intramuscular, etc.). The Example is not to be construed as limiting the scope of the invention.

### Overview of the Example

The use of perfusion machines is rapidly growing in the field of transplantation. Vascularized Composite Allotransplantations and reconstructive surgery are not to be outdone with first descriptions of machine perfusion (MP) applications. The goal is to improve preservation by continuously delivering oxygen and nutrients essential for cellular activity while clearing metabolic waste, greatly decreasing ischemic complications during preservation. By decreasing ischemia-reperfusion injuries, MP can lead to short term success and long-term outcome improvements. Oxygenation is a critical parameter to ensure tissue viability during ex-vivo MP. This work presents an innovative technology based on an oxygen-sensitive, phosphorescent metalloporphyrin, which allows continuous and non-invasive oxygen monitoring of vascularized fasciocutaneous flaps. The device comprises a transparent oxygen sensing film to be applied on the flap's skin paddle, which is probed via a small, low-energy, electronic device that provides continuous monitoring of oxygen concentration. Additionally, this oxygen-sensing technology was used to monitor the oxygen levels of the perfusate inflow in parallel with the skin paddle's oxygenation. To show the reactivity and precision of this technology, we have chosen to test it using intermittent ex-vivo perfusion of porcine fasciocutaneous flaps. We present the first proof of concept results using this technology with ex-vivo machine perfusion, providing evidence of the technology's high accuracy, reactivity, and reliability, as well as its ease of use.

In this Example, we describe the use of a novel device that allows non-invasive, continuous, and responsive measurement of tissue oxygen levels in porcine vascularized fasciocutaneous flaps during ex-vivo perfusion. This Example serves as proof of concept and offers a glimpse of continuous and reliable monitoring during ex-vivo perfusion of VCAs and other organs. This technology also appears to be a solution that can be used in clinical routine for current reconstructive surgery techniques, optimizing the monitoring of microsurgical flaps.

### Materials And Methods

### Animals And Surgical Procedure

All animal care and procedures were approved by the local Institutional Animal Care and Use Committee (IACUC). Four 30 to 35 kgs female Yorkshire Pigs were included in the study of this Example. Animals were housed by the local Center for Comparative Medicine (CCM), with access to food and water, according to the Institutional Animal Care and Use Committee (IACUC) guidelines. Surgery was performed after at least 24 hours of acclimation. Animals were sedated, intubated, and maintained under general anesthesia during the whole procedure by veterinarians. Continuous monitoring included heart rate, electrocardiography temperature, respiratory rate, and oxygen saturation. After a single systemic heparin injection (100 UI/kg), the saphenous fasciocutaneous flap was harvested from the right groin of the animal. The flap limits and anatomic markings were drawn to allow an elliptical skin paddle with a short and long axis of 5.5 and 9 cm before incision, respectively. The surgical technique has been previously described by our team [Ref. 21]. The saphenous flap was harvested on the femoral vessels, allowing smooth cannulation of the artery and easy procurement of the outflow from the vein receiving two venae comitantes from the flap. Following meticulous dissection, the distal femoral vessels were ligated, and the flap was freed after division of the proximal portion. The vascularized flap was then flushed using 4 °C heparinized saline until clear outflow. Permission from the IACUC could have been granted for on-table transfers, allowing other groups to harvest organs and tissues to optimize the number of animals used in the institution. Animals were euthanized at the end of the procedure.

### Fasciocutaneous Flap Ex-Vivo Perfusion

Immediately after flushing, the flap was transported under a Class II biosafety hood. The 18G arterial cannula was linked to a customized machine perfusion system (FIG. 1: machine perfusion setup). A hollow fiber oxygenator (Affinity Pixie, Medtronic, Dublin, Ireland) was connected to an oxygen tank (95% O₂, 5% CO₂), which allowed for oxygenation of the circulating perfusate. A pressure transducer was incorporated into the closed system and allowed for continuous monitoring of the pressure. A modified Steen solution was used as a perfusate. The perfusate was circulating through the flap's vascular tree, and the outflow was sampled through the cannulated femoral vein. The perfusate was recirculated and a full perfusate exchange was performed every 24 hours if needed. A total volume of 350 ml was circulating. Sodium bicarbonate could be used to correct the pH. The initial flow rate was based on our experience and perioperative color Doppler velocity measurements performed intraoperatively [Ref. 22]. The perfusion rhythm was intermittent: The flap was perfused between 30 and 45 minutes, followed by an ischemic period of 75 to 90 minutes. The roller pump used in the perfusion system (DRIVE MASTERFLEX L/S, Cole-Parmer, Vemnon Hills, Illinois, USA) allowed for programming the cycles. The delivered flow was nonpulsatile. Perfusate samples were procured frequently and included blood gas analyses (BGA) performed with a clinical-grade measurement system (Rapidpoint 500, Siemens, Munich, Germany). The perfusion was terminated if the weight gain reached 50% of the initial weight, or if the vascular resistance was too high to allow a flow F≥50% of the initial value.

### Oxygen Monitoring

The technology used in this Example is based on the detection of changes in the phosphorescence lifetime and intensity of an ultrabright metalloporphyrin molecules [Ref. 23], which exhibit oxygen quenching of phosphorescence. The change in the phosphor's lifetime rand intensity I depends on oxygen following the Stern-Volmer relation [Ref. 24]. This phosphor can be readily embedded within polymer-based films, resulting in ultra-thin, breathable films that exhibit bright emission through the oxygen partial pressure (pO₂) range 0-760 mmHg, and are impervious to changes in relative humidity [Ref. 25, 26].

For this Example, we employed the oxygen sensors for two applications, sensing of transcutaneous oxygen tension and of partial oxygen pressure in the perfusate.

To sense pO₂ via skin, we designed an adhesive, medical grade film [Ref. 27] which were applied to the skin paddle of the flap after rigorous drying. The oxygen sensing film is composed of three layers: (i) a medical-grade semi-permeable transparent membrane (Bioclusive, Mckesson), which partially blocks atmospheric oxygen from reaching the skin; (ii) a thin poly(propyl methacrylate) (PPMA) layer with the embedded metalloporphyrins; (iii) a highly breathable white scattering layer which both enhances the collection of the phosphorescence and acts as optical insulation, improving the estimation of oxygenation and preventing external light interference.

Oxygen partial pressure in the perfusate fluid is carried out with a 3D-printed flow cell containing the same polymer film and scattering layer in its top lining. A breathable layer of medical adhesive (3M) covers the scattering layer and acts as a physical barrier between the O₂-sensing film and the circulating perfusate. The flow cell monitoring the perfusate inflow oxygenation was placed 4 cm upstream of the arterial cannula. Outflow oxygen was measured when possible, placing the flow cell 4 cm downstream the cannulated femoral vein.

Changes in the phosphorescence of the O₂-sensing layers are detected by a portable sensor, initially designed as a wearable transcutaneous oxygenation monitoring (TCOM) device [Ref. 27]. The prototype device's sensor head is affixed to the films or flow cells centered over the O₂-sensing layer, exciting the phosphorescence of the porphyrins via two ultraviolet LEDs, with the phosphorescence detected via a small photodiode. A small temperature sensor (thermistor) in the sensor head allows us to account for temperature-dependent effects in the phosphorescence and to produce a temperature compensated pO₂ reading.

The sensor head is linked to the main control electronics, built around a commercially available microcontroller board (Particle Photon), which is then connected to a laptop through a USB serial port (FIG. 1: Setup of the device on the flap+composition of the device). The devices allow us to continuously monitor oxygen levels of the tissue underneath (TcPO₂) and the perfusate during experiments lasting multiple days, with a chosen sampling time of 30 seconds. The continuous oxygenation readings from all devices were saved and displayed real-time, in units of mmHg, via a Python [Ref. 28] script on a PC through USB serial port. Oxygenation readings are obtained from the phosphorescence by previously calibrating the response of the materials and fitting the resulting dependence to pO₂ with the Stern-Volmer relation.

### Results

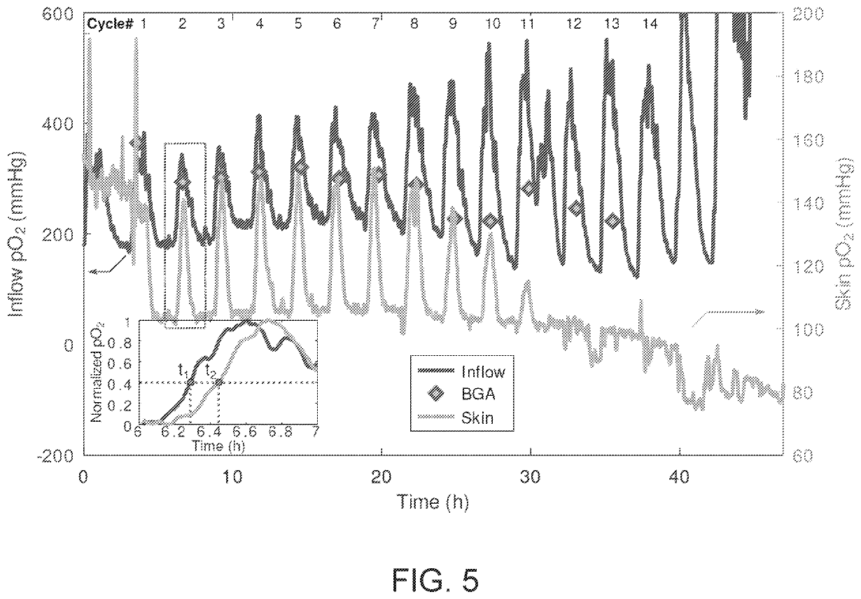

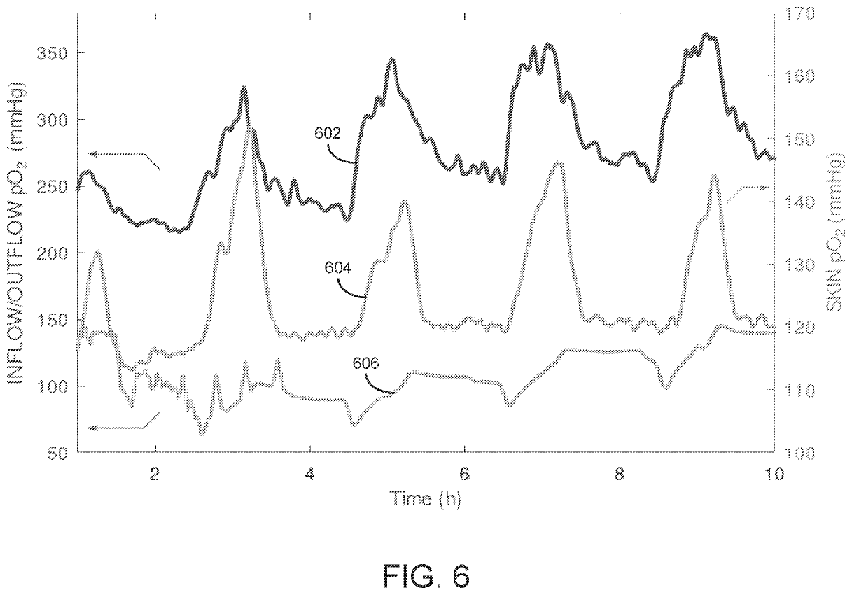

We were able to simultaneously monitor the oxygen levels of the perfusate inflow in parallel with the skin paddle's oxygenation in multiple experiments. FIG. 5, plots the results from an experiment in which two devices were used, showing continuous readings of the circulating oxygen (PO₂) in the inflow and the skin oxygen levels (TcPO₂, the right Y axis). In experiments in which the femoral vein could also be cannulated, we were able to monitor from an inflow gas sensor 602, sample gas sensor 604, and outflow gas sensor 606 to include outflow PO₂ as well, with results shown in FIG. 6.

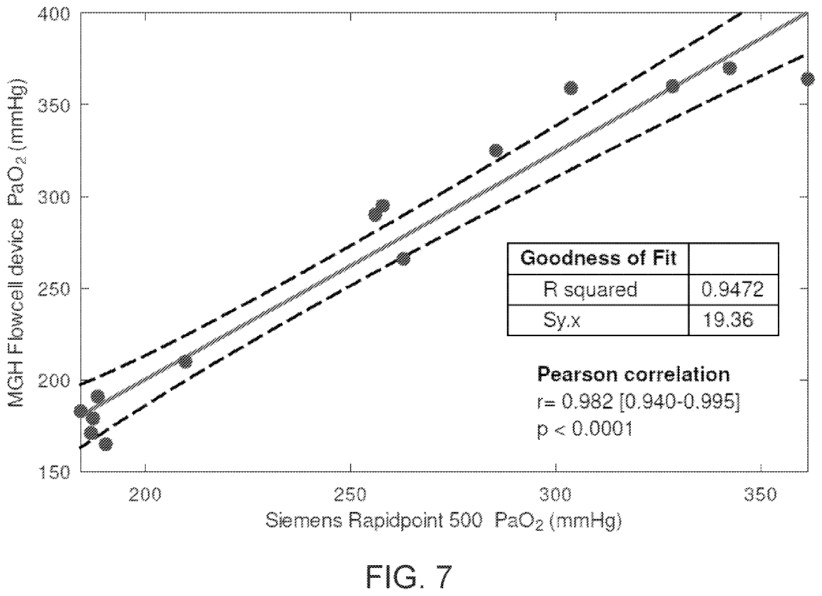

To assess the values provided by the flowcell-based devices in circulating oxygen values, a correlation test was performed. It should be noted that the calibrated device incorporated in the perfusion system provided continuous values while the conventional blood gas analyses were performed on repeated perfusate samples. In order to compare the continuous and discrete variable, a 5 minute average value of the flowcell readings was calculated to compare to each BGA measurement. FIG. 7 shows the correlation plot and the Pearson correlation coefficient, where a statistically significant correlation was found between the measurements (r=0.982, p<0.001).

Four ex-vivo perfused saphenous fasciocutaneous flaps were included in the statistical analysis described below. The mean warm ischemia time was 13.4±2.7 minutes. The mean time-to-stabilization of the flow was 33.8±21.4 minutes. After this initial phase where the flow was adapted to the vascular resistances, the flaps underwent intermittent perfusion. The first two flaps underwent 30 minutes of perfusion (ON) followed by 90 minutes of ischemia (OFF). The perfusion rhythm was changed to 30 minutes ON/75 minutes OFF. The mean total perfusion time was 50 hours (range 28-76 hours). The mean total number of perfusion cycles per flap was 19.0±5.5, for a total of 76 cycles. We included the following variables in the statistical analysis obtained from each cycle: the maximum (Max) and minimum (Min) oxygen values in both skin (TcPO₂) and in the inflow perfusate (PO₂) upstream the arterial cannula; the difference Δ=Max−Min for TcPO₂ and PO₂ for each cycle; the time delay between the measured inflow PO₂ and TcPO₂. The delay was calculated by normalizing the inflow PO₂ and TcPO₂ between [0,1], and finding the time difference between both curves reaching a value of 0.4 (see inset of FIG. 5).

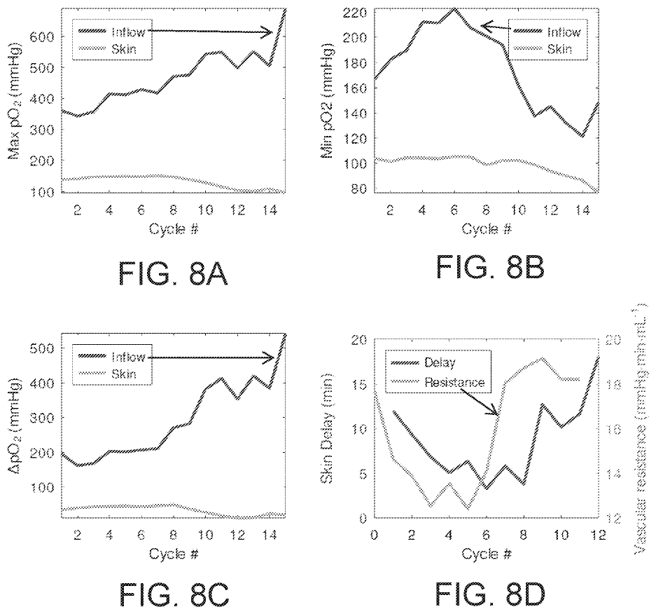

The described variables, extracted from the measurement in FIG. 6, are shown in FIGS. 8A-8D, where the following features are observed:
Both the maximum and minimum TcPO₂ experience a sharp drop following cycle #8.
Additionally, the difference between PO₂ and TcPO₂ increases at the end of the perfusion, corresponding with perfusion failure backed by substantial increase in vascular resistance.
The delay between circulating inflow PO₂ and skin TcPO₂ shows a sharp increase following cycle #8, which was also correlated to increasing vascular resistance (FIG. 8D).

### Discussion

One objective of this Example was to test the reactivity and reliability of continuous oxygen readings using a novel technology. The vascularized saphenous flaps used in the included experiments allowed testing circulating oxygen values, relevant to all machine-perfusion based approaches. The perfusion design with interruptions of 75 to 90 minutes, followed by active oxygenation of 30 to 45 minutes allowed periodically assessing the response during the experiments, providing a total of 76 cycles used as replicates. The proposed system was easily implemented in a custom-made perfusion system, and the user-friendly computer interface made it possible to be used by a non-expert surgical team.

To control effective oxygenation during machine perfusion, a measurement flow cell was placed upstream the arterial cannula, providing continuous circulating oxygen values (PO₂) delivered to the perfused organ. To our knowledge, no clinically used perfusion system is currently using such a monitoring. Oxygen is critical for ATP production in the mitochondria. While ischemia is almost inevitable during a transplantation procedure, the recent advances in machine perfusion have led to massive improvement in decreasing the total ischemic time. However, most of the injuries are provoked during the reperfusion process, by restoring high oxygen levels after a metabolic shutdown. So far, transplant teams have no target oxygen levels to halt the progression of ischemia-reperfusion injury (IRI) before the generation of reactive oxygen species (ROS).

If machine perfusion seems to be a major step towards improving transplantation outcomes, this limitation leaves clinicians with the challenge of mitigating its consequences. The challenge of IRI and ROS is problematic especially for extended criteria donor organs. The molecular mechanisms occurring during machine perfusion are not yet fully comprehended, and even if some teams are actively studying this specific field, they are still limited by the need for repeated blood gas measurements. Moreover, machine perfusion is also used to assess organ function. Oxygen consumption is a critical indicator of cell, tissue and organ activity, and can be determined by implementing the arteriovenous oxygen difference, the flowrate, and the weight.

Being able to continuously measure the oxygen values in both the inflow and outflow perfusate, the method described in our study opens the door to live O₂ consumption monitoring in addition to the level of tissue oxygenation provided by inflow and tissue oxygen readings.

A delay was found between changes in measured PO₂ and TcPO₂. This was expected because of the time needed for the perfusate to reach the distal capillaries of the flap. Interestingly, this delay correlated with vascular resistance, which were monitored continuously. However, only a few timepoints were assessed, and further research is needed to prove replicability and fully explore and harness the potential of this feature.

This Example also proved reliable transcutaneous oxygenation readings in fasciocutaneous flaps. The saphenous flap model is relevant for the skin component of VCAs. By continuously monitoring tissue oxygenation of these complex organs (such as face or band transplants), reconstructive surgeons could substantially improve the outcomes of these difficult procedures marked by long term immune rejection. IRI are indeed known to be implicated in VCA rejection by increasing antigen release in a highly immunogenic tissue such as skin. Similar to the potential contributions in the field of solid organ transplantation, the technology could be precious in these reconstructive transplants. The model used is also directly relevant for wide autologous reconstructive surgery involving flap transfer and monitoring. Fasciocutaneous flaps have become the gold standard in complex reconstructions over the last decade. Although the techniques are increasingly improved and mastered, a failure rate persists for pedicled and free flaps (2 to 10% according to authors). The delay of surgical revision is a significant element in the case of vascularization dysfunction of flaps. The precocity of revision is correlated with the flap salvage rate. So far, flap monitoring is mainly clinical by assessing color, skin capillary refill time (CRT), and overall appearance. Those parameters show poor reactivity, since paleness, coldness and increase of the CRT occur late after acute vascular complications, highly compromising the flap survivability and salvage feasibility. Some authors described oxygen monitoring using Near-Infrared Spectroscopy (NIRS) and LiCox probes (Integra Lifescience, Princetown, New Jersey, USA) [Ref. 19, 29, 30]. NIRS has the disadvantage of being discontinuous and hemoglobin dependent, while the diverted use of LiCox is invasive and highly susceptible to movement, using bulky and expensive equipment. Moreover, the simplicity of implementation and the readability of the measurements must be appropriate to allow efficiency in the clinical routine. The technology we describe in this Example allows live monitoring in a precise and reactive way of the oxygenation of fasciocutaneous flaps. This opens perspectives of efficient monitoring, with the possibility of activating an alarm when the oxygen level drops below a defined threshold postoperatively in order to alert the teams and optimize the chances of survival of the flap after surgical revision. However, the transcutaneous oxygenation measurement presented herein is influenced by atmospheric oxygen and provides readings which are slightly above tissue oxygen concentration. The true TcpO₂ can be calculated via mathematical models [Ref. 31]. It is contemplated that one can implement such calculations in device firmware to provide live readings of true tissue oxygenation. This non-invasive tissue monitoring technique, whose technology has been validated in another form for monitoring Deep Inferior Epigastric Perforator (DIEP) flaps, can be used for all free or pedicled flaps with a skin component. In addition, the early applications of machine perfusion in autologous procedures open new horizons in reconstructive surgery, as shown by the techniques developed by Wolff et al. [Ref. 32, 33]. They achieved free flap reconstruction with no anastomoses, by perfusing the transferred free flap with diluted compatible blood. Their innovative technique was limited by ischemic complications. In the field, no technology has been used to continuously monitor perfused organs in clinical transplantation. In autologous reconstructive surgery, some authors have described the use of devices measuring oxygen within skin flaps to detect early signs of vascular failure leading to effective reintervention [Ref. 18, 19]. However, the technology used is often hemoglobin-dependent, while most work on ex-vivo perfusion including VCAs uses acellular perfusates [Ref. 1, 15, 20], making these readings ineffective. Our team showed in this Example that extended perfusion of fasciocutaneous flaps can be performed using an acellular perfusate solution. The idea of a universal monitoring system that can be used regardless of the perfusion or temperature conditions would increase the safety of these novel procedures and help overcome the limits currently facing plastic surgeons.

### REFERENCES

(1) van Beekum, C. J.; Vilz, T. O.; Glowka, T. R.; von Websky, M. W.; Kalff, J. C.; Manekeller, S., “Normothermic machine perfusion (NMP) of the liver-current status and future perspectives”, Annals of Transplantation 2021, 26, e931664-1.
(2) Czigany, Z.; Pratschke, J., Fronek, J.; Guba, M.; Schoning, W.; Raptis, D. A.; Andrassy, J.; Kramer, M.; Strnad, P.; Tolba, R. H., et al., “Hypothermic oxygenated machine perfusion reduces early allograft injury and improves post-transplant outcomes in extended criteria donation liver transplantation from donation after brain death: results from a multicenter randomized controlled trial (HOPE ECD-DBD)”, Annals of Surgery 2021, 274, 705-712.
(3) MacConmara, M.; Hanish, S. I.; Hwang, C. S.; De Gregorio, L.; Desai, D. M.; Feizpour, C. A.; Tanriover, B.; Markmann, J. F.; Zeh III, H.; Vagefi, P. A., “Making every liver count: increased transplant yield of donor livers through normothermic machine perfusion”, Annals of Surgery 2020, 272, 397-401.
(4) Qin, G.; Jernryd, V.; Sjoberg, T.; Steen, S.; Nilsson, J., “Machine perfusion for human heart preservation: a systematic review”, Transplant International 2022, 35, 10258.
(5) Markmann, J. F.; Abouljoud, M. S.; Ghobrial, R. M.; Bhati, C. S.; Pelletier, S. J.; Lu, A. D.; Ottmann, S.; Klair, T.; Eymard, C.; Roll, G. R., et al., “Impact of portable normothermic blood-based machine perfusion on outcomes of liver transplant: the OCS liver PROTECT randomized clinical trial”, JAMA Surgery 2022, 157, 189-198.
(6) Husen, P.; Boffa, C.; Jochmans, I.; Krikke, C.; Davies, L.; Mazilescu, L.; Brat, A.; Knight, S.; Wettstein, D.; Cseprekal, O., et al., “Oxygenated end-hypothermic machine perfusion in expanded criteria donor kidney transplant: a randomized clinical trial”, JAMA Surgery 2021, 156, 517-525.
(7) Tawa, P.; Goutard, M.; Lupon, E.; Tratnig-Frankl, P.; Lellouch, A. G.; Cetrulo Jr, C. L., Reconstructive Transplantation; Springer, 2023; pp 231-238.
(8) Caplan, A. L.; Parent, B.; Kahn, J.; Dean, W.; Kimberly, L. L.; WP, A. L.; Rodriguez, E. D., et al., “Emerging ethical challenges raised by the evolution of vascularized composite allotransplantation”, Transplantation 2019, 103, 1240-1246.
(9) Lantieri, L.; Cholley, B.; Lemogne, C.; Guillemain, R.; Ortonne, N.; Grimbert, P.; Thervet, E.; Lellouch, A. G., “First human facial retransplantation: 30-month follow-up”, The Lancet 2020, 396, 1758-1765.
(10) Haug, V.; Kollar, B.; Obed, D.; Kiwanuka, H.; Turk, M.; Wo, L.; Tasigiorgos, S.; Kueckelhaus, M.; Riella, L. V.; Pomahac, B., “The evolving clinical presentation of acute rejection in facial transplantation”, JAMA Facial Plastic Surgery 2019, 21, 278-285.
(11) Krezdorn, N.; Lian, C. G.; Wells, M.; Wo, L.; Tasigiorgos, S., Xu, S.; Borges, T. J.; Frierson, R. M.; Stanek, E.; Riella, L. V., et al., “Chronic rejection of human face allografts”, American Journal of Transplantation 2019, 19, 1168-1177.
(12) He, J., Khan, U. Z., Qing, L.; Wu, P.; Tang, J., “Improving the ischemia-reperfusion injury in vascularized composite allotransplantation: Clinical experience and experimental implications”, Frontiers in Immunology 2022, 13, 998952.
(13) Huang, C. A.; Wang, Z.; CO, C. U. A., “Investigating Novel Approaches to Block Inflammation and Prevent Ischemia Reperfusion Injury During VCA Transplantation. 2020.
(14) Kruit, A. S., Smits, L.; Pouwels, A.; Schreinemachers, M.-C. J.; Hummelink, S. L.; Ulrich, D. J., “Ex-vivo perfusion as a successful strategy for reduction of ischemia-reperfusion injury in prolonged muscle flap preservation—a gene expression study”, Gene 2019, 701, 89-97.
(15) Goutard, M.; de Vries, R. J.; Tawa, P.; Pendexter, C. A.; Rosales, I. A.; Tessier, S. N., Burlage, L. C.; Lantieri, L.; Randolph, M. A.; Lellouch, A. G., et al., “Exceeding the limits of static cold storage in limb transplantation using subnormothermic machine perfusion”, Journal of Reconstructive Microsurgery 2023, 39, 350-360
(16) Berkane, Y.; Lellouch, A. G., Shamlou, A. A.; Goutard, M.; Tawa, P.; Uygun, B. E.; Randolph, M. A.; Cetrulo Jr, C. L.; Uygun, K., “121. Acellular Subnomothermic Machine Perfusion of Fasciocutaneous Flaps in Swine”, Plastic and Reconstructive Surgery Global Open 2023, 11.
(17) Hofmann, J.; Otarashvili, G.; Meszaros, A.; Ebner, S.; Weissenbacher, A.; Cardini, B.; Oberhuber, R.; Resch, T.; “Ofner, D.; Schneeberger, S., et al., “Restoring mitochondrial function while avoiding redox stress: the key to preventing ischemia/reperfusion injury in machine perfused liver grafts?”, International Journal of Molecular Sciences 2020, 21, 3132.
(18) Kohler, L. H.; Kohler, H.; Kohler, S.; Langer, S.; Nuwayhid, R.; Gockel, I., Spindler, N.; Osterhoff, G., “Hyperspectral Imaging (HSI) as a new diagnostic tool in free flap monitoring for soft tissue reconstruction: A proof of concept study”, BMC surgery 2021, 21, 222.
(19) Arnez, Z. M.; Ramella, V.; Papa, G.; Novati, F. C.; Manca, E.; Leuzzi, S.; Stocco, C., “Is the LICOX® PtO2 system reliable for monitoring of free flaps? Comparison between two cohorts of patients”, Microsurgery 2019, 39, 423-427.
(20) Kruit, A. S.; Schreinemachers, M.-C. J.; Koers, E. J.; Zegers, H. J.; Hummelink, S.; Ulrich, D. J., “Successful longterm extracorporeal perfusion of free musculocutaneous flaps in a porcine model”, Journal of Surgical Research 2019, 235, 113-123.
(21) Pozzo, V.; Romano, G.; Goutard, M.; Lupon, E.; Tawa, P.; Acun, A.; Andrews, A. R., Taveau, C. B.; Uygun, B. E.; Randolph, M. A., et al., “A reliable porcine fasciocutaneous flap model for vascularized composite allografts bioengineering studies”, JoVE (Journal of Visualized Experiments) 2022, e63557.
(22) Goudot, G.; Berkane, Y.; de Clermont-Tonnerre, E.; Guinier, C.; Filz von Reiterdank, I.; van Kampen, A.; Uygun, K.; Cetrulo Jr. C. L.; Uygun, B. E.; Dua, A., et al., “Microvascular assessment of fascio-cutaneous flaps by ultrasound: A large animal study”, Frontiers in Physiology 2022, 13, 2592.
(23) Roussakis, E.; Li, Z.; Nowell, N. H.; Nichols, A. J.; Evans, C. L., “Bright, “clickable” porphyrins for the visualization of oxygenation under ambient light”, Angewandte Chemie International Edition 2015, 54, 14728-14731.
(24) Stern, O.; Volmer, M., “Uber die abklingzeit der fluoreszenz”, Phys. Z 1919, 20, 183-188.
(25) Roussakis, E.; Cascales, J. P.; Marks, H. L., Li, X.; Grinstaff, M.; Evans, C. L., “Humidity-Insensitive Tissue Oxygen Tension Sensing for Wearable Devices”, Photochemistry and Photobiology 2020, 96, 373-379.
(26) Li, X.; Roussakis, E.; Cascales, J. P.; Marks, H. L.; Witthauer, L., Evers, M.; Manstein, D.; Evans, C. L., “Optimization of bright, highly flexible, and humidity insensitive porphyrin-based oxygen-sensing materials”, Journal of Materials Chemistry C 2021, 9, 7555-7567.
(27) Cascales, J. P.; Roussakis, E.; Witthauer, L.; Goss, A.; Li, X.; Chen, Y.; Marks, H. L.; Evans, C. L., “Wearable device for remote monitoring of transcutaneous tissue oxygenation”, Biomed. Opt. Express 2020, 11, 6989-7002.
(28) Van Rossum, G.; Drake, F. L., The Python language reference manual; Network Theory Ltd., 2011.
(29) Repez, A.; Oroszy, D.; Arnez, Z. M. “Continuous postoperative monitoring of cutaneous free flaps using near infrared spectroscopy”, Journal Of Plastic, Reconstructive & Aesthetic Surgery 2008, 61, 71-77.
(30) Newton, E.; Butskiy, O.; Shadgan, B.; Prisman, E.; Anderson, D. W., “Outcomes of free flap reconstructions with near-infrared spectroscopy (NIRS) monitoring: A systematic review”, Microsurgery 2020, 40, 268-275.
(31) Cascales, J. P.; Draghici, A. E.; Keshishian, H.; Taylor, J. A.; Evans, C. L., “Calculation of Tissue Oxygenation via an Inverse Boundary Problem for Transcutaneous Oxygenation Wearable Applications”, ACS Measurement Science Au 2023.
(32) Wolff, K.-D.; Mucke, T.; von Bomhard, A.; Ritschl, L. M.; Schneider, J.; Humbs, M.; Fichter, A. M., “Free flap transplantation using an extracorporeal perfusion device: First three cases”, Journal of Cranio-Maxillofacial Surgery 2016, 44, 148-154.
(33) Wolff, K.-D., “New aspects in free flap surgery: Miniperforator flaps and extracorporeal flap perfusion”, Journal Of Stomatology, Oral And Maxillofacial Surgery 2017, 118, 238-241.
   The citation of any document is not to be construed as an admission that it is prior art with respect to the present invention.

Thus, the invention provides improved perfusion systems for a tissue sample.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A perfusion system for a tissue sample, the system comprising:
a perfusion fluid source;
an inflow conduit in fluid communication with the perfusion fluid source and the sample, the inflow conduit being configured to deliver perfusion fluid to the sample;
an outflow conduit in fluid communication with the sample, the outflow conduit being configured to carry perfusion fluid away from the sample;
a first gas sensor in fluid communication with the inflow conduit, the first gas sensor measuring a concentration of a gas in the perfusion fluid in the inflow conduit; and
a second gas sensor contacting the sample, the second gas sensor measuring a concentration of a gas in the sample.

2. The perfusion system of claim 1 wherein the second gas sensor comprises:
a photoluminescent oxygen-sensitive probe in contact with the sample;
a photon source configured to direct photons at the photoluminescent oxygen-sensitive probe;
a photodetector configured to detect light emitted from the photoluminescent oxygen-sensitive probe when the photon source directs photons at the photoluminescent oxygen-sensitive probe; and
a controller in electrical communication with the photon source and the photodetector, the controller being configured to execute a program stored in the controller to calculate a concentration of oxygen adjacent to the photoluminescent oxygen-sensitive probe from an electrical signal received from the photodetector.

3. The perfusion system of claim 2 wherein:
the photoluminescent oxygen-sensitive probe is a formulation having an emission that provides tissue oxygen partial pressure (pO₂).

4. The perfusion system of claim 3 wherein:
the photoluminescent oxygen-sensitive probe comprises a polymeric material impregnated with a porphyrin.

5. The perfusion system of claim 3 wherein:
the photoluminescent oxygen-sensitive probe comprises a polymer impregnated with a phosphorescent meso-unsubstituted porphyrin.

6. The perfusion system of claim 1 wherein the second gas sensor comprises:
an oxygen-sensitive probe configured for insertion into the sample; and
a controller in electrical communication with the oxygen-sensitive probe, the controller being configured to execute a program stored in the controller to calculate a concentration of oxygen adjacent the oxygen-sensitive probe from an electrical signal received from the oxygen-sensitive probe.

7. The perfusion system of claim 1 wherein the second gas sensor provides tissue oxygen partial pressure (pO₂).

8. The perfusion system of claim 1 further comprising:
a controller in electrical communication with the first gas sensor and the second gas sensor, the controller being configured to execute a program stored in the controller to receive electrical signals from the first gas sensor and the second gas sensor and to calculate a concentration of oxygen adjacent the first gas sensor and/or the second gas sensor.

9. The perfusion system of claim 1, wherein the first gas sensor comprises:
a material configured to undergo a change in response to oxygen partial pressure (pO₂);
a sensor head contacting the material and configured to detect the change of the material; and
a controller in electrical communication with the sensor head, the controller being configured to execute a program stored in the controller to calculate the oxygen partial pressure adjacent the material.

10. The perfusion system of claim 9 further comprising:
a transparent membrane forming an outer layer of the material;
a layer of a polymeric material embedded with a metalloporphyrin adjacent to the transparent membrane; and
a scattering layer in contact with the layer of the polymeric material and a surface of the sample.

11. The perfusion system of claim 9 wherein the first gas sensor comprises a flow cell containing the material.

12. The perfusion system of claim 9, wherein the change in the material includes a change in phosphorescence.

13. The perfusion system of claim 9, wherein the sensor head includes a plurality of light emitting diodes (LEDs) and a photodiode.

14. The perfusion system of claim 13, wherein the sensor head further includes a temperature sensor.

15. The perfusion system of claim 1, wherein the second gas sensor comprises:
a material configured to undergo a change in response to oxygen pressure; and
a sensor head contacting the material and configured to detect the change of the material; and
a controller in electrical communication with the sensor head, the controller being configured to execute a program stored in the controller to calculate the oxygen partial pressure adjacent the material.

16. The perfusion system of claim 15, wherein in the second oxygen sensor, the material is placed on an outer surface of the sample.

17. The perfusion system of claim 15, wherein the change in the material includes a change in phosphorescence intensity or phosphorescence lifetime.

18. The perfusion system of claim 15, wherein the sensor head includes a plurality of light emitting diodes (LEDs) and a photodiode.

19. The perfusion system of claim 18, wherein the sensor head further includes a temperature sensor.

20. The perfusion system of claim 1, further comprising:
a third gas sensor in fluid communication with the outflow conduit, the third gas sensor measuring a concentration of a gas in perfusion fluid in the outflow conduit.

21. The perfusion system of claim 20, wherein the third gas sensor comprises:
a material configured to undergo a change in response to oxygen partial pressure (pO₂);
a sensor head contacting the material and configured to detect the change of the material; and
a controller in electrical communication with the sensor head, the controller being configured to execute a program stored in the controller to calculate the oxygen partial pressure adjacent to the material.

22. The perfusion system of claim 21 further comprising:
a transparent membrane forming an outer layer of the material;
a layer of a polymeric material embedded with a metalloporphyrin adjacent to the transparent membrane; and
a scattering layer in contact with the layer of the polymeric material and a surface of the sample.

23. The perfusion system of claim 21 wherein the third gas sensor includes a flow cell containing the material.

24. The perfusion system of claim 21, wherein the change in the material includes a change in phosphorescence intensity or phosphorescence lifetime.

25. The perfusion system of claim 24, wherein the sensor head includes a plurality of light emitting diodes (LEDs) and a photodiode.

26. The perfusion system of claim 25, wherein the sensor head further includes a temperature sensor.

27. The perfusion system of claim 1 further comprising:
an oxygenator in fluid communication with the perfusion fluid source; and
a pump for circulating the perfusion fluid through the oxygenator, the inflow conduit, and the outflow conduit,
wherein the oxygenator controls oxygen level in perfusion fluid flowing towards the sample.

28. The perfusion system of claim 27 further comprising:
a heat exchanger for adjusting a temperature of the perfusion fluid.

29. The perfusion system of claim 27 further comprising:
a controller in electrical communication with the oxygenator, the first gas sensor, and the second gas sensor, the controller being configured to execute a program stored in the controller to control oxygen level in perfusion fluid flowing towards the sample based on electrical signals received from the first gas sensor and the second gas sensor.

30. The perfusion system of claim 27 further comprising:
a controller in electrical communication with the pump, the first gas sensor, and the second gas sensor, the controller being configured to execute a program stored in the controller to control oxygen level in perfusion fluid flowing towards the sample based on electrical signals received from the first gas sensor and the second gas sensor.

31. The perfusion system of claim 1 further comprising:
a controller in electrical communication with the first gas sensor and the second gas sensor, the controller being configured to execute a program stored in the controller to receive electrical signals from the first gas sensor and the second gas sensor and to activate delivery of the perfusion fluid when a loss of viability is calculated based on the first concentration of the first gas adjacent the first gas sensor and the second concentration of the second gas adjacent the second gas sensor.

32. The perfusion system of claim 1 further comprising:
a controller in electrical communication with the first gas sensor and the second gas sensor, the controller being configured to execute a program stored in the controller to receive electrical signals from the first gas sensor and the second gas sensor and to deactivate delivery of the perfusion fluid when a calculated viability based on the first concentration of the first gas adjacent the first gas sensor and the second concentration of the second gas adjacent the second gas sensor exceeds a threshold viability value.

33. The perfusion system of claim 1 further comprising:
a controller in electrical communication with the first gas sensor and the second gas sensor, the controller being configured to execute a program stored in the controller to receive electrical signals from the first gas sensor and the second gas sensor and to intermittently activate or deactivate delivery of the perfusion fluid based on the first concentration of the first gas adjacent the first gas sensor and the second concentration of the second gas adjacent the second gas sensor.

34. The perfusion system of claim 1 further comprising:
a controller in electrical communication with the first gas sensor and the second gas sensor, the controller being configured to execute a program stored in the controller to activate an alarm when a gas level drops below a defined threshold based on electrical signals received from the first gas sensor and the second gas sensor.

35. The perfusion system of claim 1 wherein:
the perfusion fluid is an acellular perfusate solution.

36. The perfusion system of claim 1 wherein:
the first gas sensor provides continuous circulating oxygen values delivered to the perfused sample.

37. The perfusion system of claim 1 wherein:
the second gas sensor provides continuous tissue oxygenation values of the perfused sample.

38. A method for machine perfusion of a tissue sample, the method comprising:
(a) providing a tissue sample;
(b) delivering a perfusion fluid to the sample via an inflow conduit;
(c) measuring a concentration of a gas in the perfusion fluid in the inflow conduit; and
(d) measuring a concentration of a gas in the sample.

39. The method of claim 38 further comprising:
(e) measuring a concentration of a gas in the perfusion fluid in an outflow conduit configured to carry perfusion fluid away from the sample.

40. The method of claim 39 wherein:
step (c) comprises measuring the concentration of the gas in the perfusion fluid in the inflow conduit with a first gas sensor,
step (d) comprises measuring the concentration of the gas in the sample with a second gas sensor, and
step (e) comprises measuring the concentration of the gas in the perfusion fluid in the outflow conduit with a third gas sensor,
wherein at least one of the first gas sensor, the second gas sensor and the third gas sensor comprises an oxygen-sensitive probe.

41. The method of claim 39 wherein:
step (c) comprises measuring the concentration of the gas in the perfusion fluid in the inflow conduit with a first gas sensor,
step (d) comprises measuring the concentration of the gas in the sample with a second gas sensor; and
step (e) comprises measuring the concentration of the gas in the perfusion fluid in the outflow conduit with a third gas sensor,
wherein at least one of the first gas sensor, the second gas sensor and the third gas sensor comprises a material configured to undergo a change in response to oxygen partial pressure (pO₂).

42. The method of claim 40 wherein:
step (c) comprises sensing the concentration of the gas in the perfusion fluid in the inflow conduit with the first gas sensor and calculating, in a controller, a concentration of oxygen adjacent to the first gas sensor from an electrical signal transmitted to the controller from the first gas sensor,
step (d) comprises measuring the concentration of the gas in the sample with the second gas sensor, and calculating, in the controller, a concentration of oxygen adjacent the second gas sensor from an electrical signal transmitted to the controller from the second gas sensor and,
step (e) comprises sensing the concentration of the gas in the perfusion fluid in the outflow conduit with the third gas sensor and calculating, in a controller, a concentration of oxygen adjacent to the third gas sensor from an electrical signal transmitted to the controller from the third gas sensor.

43. The method of claim 39, further comprising:
monitoring a change in the concentration from the first gas sensor, and at least one of the second gas sensor and third gas sensor,
measuring a variation in the oxygen concentration over a period of time;
analyzing the variation in the oxygen concentration over the period of time; and
estimating a viability of the tissue sample and a perfusion quality.

44. The method of claim 38 further comprising:
(e) generating a report, using the controller, of oxygen perfusion in the sample.

45. The method of claim 38 further comprising:
(e) controlling an oxygen level in the perfusion fluid flowing towards the sample based on electrical signals received from the first gas sensor and the second gas sensor.

46. The method of claim 38 further comprising:
(e) activating an alarm when a gas level drops below a defined threshold based on electrical signals received from the first gas sensor and the second gas sensor.

47. The method of claim 38 wherein:
the tissue sample is a vascular composite allograft.

48. The method of claim 47 wherein:
the vascular composite allograft is at least a portion of a limb, face, larynx, trachea, abdominal wall, genitourinary tissue, uterine tissue, or solid organ, or any combination thereof.

49. The method of claim 38 wherein:
the tissue sample is a donor vascular composite allograft for vascular composite allograft transplantation.

50. The method of claim 38 wherein:
the tissue sample is obtained from a human, a primate, or a pig.

51. The method of claim 38 wherein:
the tissue sample is a fasciocutaneous flap.

52. A perfusion system for a tissue sample, the system comprising:
a perfusion fluid source;
an inflow conduit in fluid communication with the perfusion fluid source and the sample, the inflow conduit being configured to deliver perfusion fluid to the sample;
an outflow conduit in fluid communication with the sample, the outflow conduit being configured to carry perfusion fluid away from the sample;
a first gas sensor in fluid communication with the inflow conduit, the first gas sensor measuring a concentration of a first gas in the perfusion fluid in the inflow conduit;
a second gas sensor contacting the sample, the second gas sensor measuring a concentration of a second gas in the sample; and
a controller in electrical communication with the first gas sensor and the second gas sensor, the controller being configured to execute a program stored in the controller to:
(i) receive electrical signals from the first gas sensor and the second gas sensor and to calculate a first concentration of the first gas adjacent the first gas sensor and a second concentration of the second gas adjacent the second gas sensor, and
(ii) activate or deactivate delivery of the perfusion fluid flowing towards the sample and/or adapt perfusion parameters based on the first concentration of the first gas adjacent to the first gas sensor and the second concentration of the second gas adjacent to the second gas sensor.

53. The system of claim 52, wherein the controller is configured to execute the program stored in the controller to activate delivery of the perfusion fluid when a loss of viability is calculated based on the first concentration of the first gas adjacent the first gas sensor and the second concentration of the second gas adjacent the second gas sensor.

54. The system of claim 52, wherein the controller is configured to execute the program stored in the controller to deactivate delivery of the perfusion fluid when a calculated viability based on the first concentration of the first gas adjacent the first gas sensor and the second concentration of the second gas adjacent the second gas sensor exceeds a threshold viability value.

55. The system of claim 52, wherein the controller is configured to execute the program stored in the controller to intermittently activate or deactivate delivery of the perfusion fluid based on the first concentration of the first gas adjacent the first gas sensor and the second concentration of the second gas adjacent the second gas sensor.

56. The system of claim 52 further comprising:
a pump in electrical communication with the controller,
wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor.

57. The system of claim 52 further comprising:
an oxygenator in electrical communication with the controller,
wherein the controller is configured to execute the program stored in the controller to control oxygen level in perfusion fluid flowing towards the sample based on the first concentration of the first gas adjacent the first gas sensor and the second concentration of the second gas adjacent the second gas sensor.

58. The system of claim 52 further comprising:
a third gas sensor in fluid communication with the outflow conduit, the third gas sensor measuring a concentration of a third gas in perfusion fluid in the outflow conduit,
wherein the controller is configured to execute the program stored in the controller to activate or deactivate delivery of the perfusion fluid flowing towards the sample and/or adapt perfusion parameters based on the first concentration of the first gas adjacent the first gas sensor and the second concentration of the second gas adjacent the second gas sensor, and the third concentration of the third gas adjacent the third gas sensor.

59. The system of claim 58, wherein the controller is configured to execute the program stored in the controller to activate delivery of the perfusion fluid when a loss of viability is calculated based on the first concentration of the first gas adjacent the first gas sensor and the second concentration of the second gas adjacent the second gas sensor, and the third concentration of the third gas adjacent the third gas sensor.

60. The system of claim 58, wherein the controller is configured to execute the program stored in the controller to deactivate delivery of the perfusion fluid when a calculated viability based on the first concentration of the first gas adjacent the first gas sensor and the second concentration of the second gas adjacent the second gas sensor, and the third concentration of the third gas adjacent the third gas sensor exceeds a threshold viability value.

61. The system of claim 58, wherein the controller is configured to execute the program stored in the controller to intermittently activate or deactivate delivery of the perfusion fluid based on the first concentration of the first gas adjacent the first gas sensor and the second concentration of the second gas adjacent the second gas sensor, and the third concentration of the third gas adjacent the third gas sensor.

62. The system of claim 58 further comprising:
a pump in electrical communication with the controller,
wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor, and the third concentration of the third gas adjacent the third gas sensor.

63. The system of claim 58 further comprising:
an oxygenator in electrical communication with the controller,
wherein the controller is configured to execute the program stored in the controller to control oxygen level in perfusion fluid flowing towards the sample based on electrical signals received from the first gas sensor and the second gas sensor, and the third concentration of the third gas adjacent the third gas sensor.

64. The system of claim 52 wherein:
the controller is in electrical communication with a controllable valve in the inflow conduit, and
the controller is configured to execute the program stored in the controller to activate or deactivate delivery of the perfusion fluid by moving the controllable valve to an open position in which the perfusion fluid is delivered to the sample or a closed position in which the perfusion fluid is not delivered to the sample.

65. The system of claim 52 wherein:
the controller is in electrical communication with a controllable valve in the inflow conduit, and
the controller is configured to execute the program stored in the controller to control delivery of the perfusion fluid by moving the controllable valve to a fully open position in which a first amount of the perfusion fluid is delivered to the sample, or an intermediate position in which a second amount of the perfusion fluid less than the first amount of the perfusion fluid is delivered to the sample, or a closed position in which the perfusion fluid is not delivered to the sample.

66. A method for machine perfusion of a tissue sample, the method comprising:
(a) providing a tissue sample;
(b) delivering a perfusion fluid to the sample via an inflow conduit;
(c) measuring a first concentration of a first gas in the perfusion fluid in the inflow conduit;
(d) measuring a second concentration of a second gas in the sample; and
(e) activating or deactivating delivery of the perfusion fluid to the sample and/or adapting perfusion parameters based on the first concentration of the first gas and the second concentration of the second gas.

67. The method of claim 66, wherein step (e) comprises activating delivery of the perfusion fluid when a loss of viability is calculated based on the first concentration of the first gas adjacent the first gas sensor and the second concentration of the second gas adjacent the second gas sensor.

68. The method of claim 66, wherein step (e) comprises deactivating delivery of the perfusion fluid when a calculated viability based on the first concentration of the first gas adjacent the first gas sensor and the second concentration of the second gas adjacent the second gas sensor exceeds a threshold viability value.

69. The method of claim 66, wherein step (e) comprises intermittently activating or deactivating delivery of the perfusion fluid based on the first concentration of the first gas adjacent the first gas sensor and the second concentration of the second gas adjacent the second gas sensor.

70. The method of claim 66, wherein step (e) comprises adjusting a flow rate of perfusion fluid flowing towards the sample based on the first concentration of the first gas adjacent the first gas sensor and the second concentration of the second gas adjacent the second gas sensor.

71. The method of claim 66, wherein step (e) comprises controlling oxygen level in perfusion fluid flowing towards the sample based on the first concentration of the first gas adjacent the first gas sensor and the second concentration of the second gas adjacent the second gas sensor.

72. A perfusion system for a tissue sample, the system comprising:
a perfusion fluid source;
an inflow conduit in fluid communication with the perfusion fluid source and the sample, the inflow conduit being configured to deliver perfusion fluid to the sample;
an outflow conduit in fluid communication with the sample, the outflow conduit being configured to carry perfusion fluid away from the sample;
a first gas sensor in fluid communication with the inflow conduit, the first gas sensor measuring a concentration of a first gas in the perfusion fluid in the inflow conduit;
a second gas sensor in fluid communication with the outflow conduit, the second gas sensor measuring a concentration of a second gas in perfusion fluid in the outflow conduit; and
a controller in electrical communication with the first gas sensor and the second gas sensor, the controller being configured to execute a program stored in the controller to: (i) receive electrical signals from the first gas sensor and the second gas sensor and to calculate a first concentration of the first gas adjacent the first gas sensor and a second concentration of the second gas adjacent the second gas sensor, and (ii) activate or deactivate delivery of the perfusion fluid flowing towards the sample and/or adapt perfusion parameters based on the first concentration of the first gas adjacent to the first gas sensor and the second concentration of the second gas adjacent to the second gas sensor.

73. The system of claim 72, wherein the controller is configured to execute the program stored in the controller to activate delivery of the perfusion fluid when a loss of viability is calculated based on the first concentration of the first gas adjacent the first gas sensor and the second concentration of the second gas adjacent the second gas sensor.

74. The system of claim 72, wherein the controller is configured to execute the program stored in the controller to deactivate delivery of the perfusion fluid when a calculated viability based on the first concentration of the first gas adjacent the first gas sensor and the second concentration of the second gas adjacent the second gas sensor exceeds a threshold viability value.

75. The system of claim 72, wherein the controller is configured to execute the program stored in the controller to intermittently activate or deactivate delivery of the perfusion fluid based on the first concentration of the first gas adjacent the first gas sensor and the second concentration of the second gas adjacent the second gas sensor.

76. The system of claim 72 further comprising:
a pump in electrical communication with the controller,
wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor.

77. The system of claim 72 further comprising:
an oxygenator in electrical communication with the controller,
wherein the controller is configured to execute the program stored in the controller to control oxygen level in perfusion fluid flowing towards the sample based on the first concentration of the first gas adjacent the first gas sensor and the second concentration of the second gas adjacent the second gas sensor.

78. The system of claim 72 further comprising:
a third gas sensor contacting the sample, the third gas sensor measuring a concentration of a third gas in the sample,
wherein the controller is configured to execute the program stored in the controller to activate or deactivate delivery of the perfusion fluid flowing towards the sample and/or adapt perfusion parameters based on the first concentration of the first gas adjacent the first gas sensor and the second concentration of the second gas adjacent the second gas sensor, and the third concentration of the third gas adjacent the third gas sensor.

79. The system of claim 78, wherein the controller is configured to execute the program stored in the controller to activate delivery of the perfusion fluid when a loss of viability is calculated based on the first concentration of the first gas adjacent the first gas sensor and the second concentration of the second gas adjacent the second gas sensor, and the third concentration of the third gas adjacent the third gas sensor.

80. The system of claim 78, wherein the controller is configured to execute the program stored in the controller to deactivate delivery of the perfusion fluid when a calculated viability based on the first concentration of the first gas adjacent the first gas sensor and the second concentration of the second gas adjacent the second gas sensor, and the third concentration of the third gas adjacent the third gas sensor exceeds a threshold viability value.

81. The system of claim 78, wherein the controller is configured to execute the program stored in the controller to intermittently activate or deactivate delivery of the perfusion fluid based on the first concentration of the first gas adjacent the first gas sensor and the second concentration of the second gas adjacent the second gas sensor, and the third concentration of the third gas adjacent the third gas sensor.

82. The system of claim 78 further comprising:
a pump in electrical communication with the controller,
wherein the controller is configured to execute the program stored in the controller to adjust a flow rate of perfusion fluid flowing towards the sample based on the first concentration of the first gas adjacent the first gas sensor and the second concentration of the second gas adjacent the second gas sensor, and the third concentration of the third gas adjacent the third gas sensor.

83. The system of claim 78 further comprising:
an oxygenator in electrical communication with the controller,
wherein the controller is configured to execute the program stored in the controller to control oxygen level in perfusion fluid flowing towards the sample based on electrical signals received from the first gas sensor and the second gas sensor, and the third concentration of the third gas adjacent the third gas sensor.

84. The system of claim 72 wherein:
the controller is in electrical communication with a controllable valve in the inflow conduit, and
the controller is configured to execute the program stored in the controller to activate or deactivate delivery of the perfusion fluid by moving the controllable valve to an open position in which the perfusion fluid is delivered to the sample or a closed position in which the perfusion fluid is not delivered to the sample.

85. The system of claim 72 wherein:
the controller is in electrical communication with a controllable valve in the inflow conduit, and
the controller is configured to execute the program stored in the controller to control delivery of the perfusion fluid by moving the controllable valve to a fully open position in which a first amount of the perfusion fluid is delivered to the sample, or an intermediate position in which a second amount of the perfusion fluid less than the first amount of the perfusion fluid is delivered to the sample, or a closed position in which the perfusion fluid is not delivered to the sample.

86. A method for machine perfusion of a tissue sample, the method comprising:
(a) providing a tissue sample;
(b) delivering a perfusion fluid to the sample via an inflow conduit;
(c) measuring a first concentration of a first gas in the perfusion fluid in the inflow conduit;
(d) measuring a second concentration of a second gas in an outflow conduit; and
(e) activating or deactivating delivery of the perfusion fluid to the sample and/or adapting perfusion parameters based on the first concentration of the first gas and the second concentration of the second gas.

87. The method of claim 86, wherein step (e) comprises activating delivery of the perfusion fluid when a loss of viability is calculated based on the first concentration of the first gas adjacent the first gas sensor and the second concentration of the second gas adjacent the second gas sensor.

88. The method of claim 86, wherein step (e) comprises deactivating delivery of the perfusion fluid when a calculated viability based on the first concentration of the first gas adjacent the first gas sensor and the second concentration of the second gas adjacent the second gas sensor exceeds a threshold viability value.

89. The method of claim 86, wherein step (e) comprises intermittently activating or deactivating delivery of the perfusion fluid based on the first concentration of the first gas adjacent the first gas sensor and the second concentration of the second gas adjacent the second gas sensor.

90. The method of claim 86, wherein step (e) comprises adjusting a flow rate of perfusion fluid flowing towards the sample based on the first concentration of the first gas adjacent the first gas sensor and the second concentration of the second gas adjacent the second gas sensor.

91. The method of claim 86, wherein step (e) comprises controlling oxygen level in perfusion fluid flowing towards the sample based on the first concentration of the first gas adjacent the first gas sensor and the second concentration of the second gas adjacent the second gas sensor.
